(19) United States
(12) Patent Application Publication
(10) Pub. No.: US 2022/0305076 A1
(43) Pub. Date: Sep. 29, 2022

(54) TOPICAL CYCLOSPORINE FOR TREATING PSORIASIS AND OTHER AILMENTS

(71) Applicant: Dyve Biosciences, Inc., Camarillo, CA (US)

(72) Inventors: Ryan BEAL, Camarillo, CA (US); Nathan FITZSIMMONS, Newbury Park, CA (US); Audrene Rice, Ventura, CA (US); Brandon SAND, Camarillo, CA (US); Kilmar MARTINEZ, Camarillo, CA (US)

(21) Appl. No.: 17/839,323

(22) Filed: Jun. 13, 2022

Related U.S. Application Data
(63) Continuation of application No. PCT/US2020/067041, filed on Dec. 24, 2020.
(60) Provisional application No. 62/953,409, filed on Dec. 24, 2019.

(51) Int. Cl. A61K 38/13; A61K 9/00; A61K 47/44; A61P 17/06; A61K 31/59; A61K 31/122; A61K 31/07; A61K 45/06; A61P 19/02; A61K 9/06

(52) U.S. Cl. CPC A61K 38/13; A61K 9/0014; A61K 47/44; A61P 17/06; A61K 31/59; A61K 31/122; A61K 31/07; A61K 45/06; A61P 19/02; A61K 9/06

(57) ABSTRACT

Disclosed herein is a transdermal delivery formulation for transdermal delivery of cyclosporine with or without one or more additional active agents through the dermis, including the skin, nail or hair follicle of a subject. The formulation overcomes the limitations of oral administration. Specifically, embodiments include a formulation and method of delivering cyclosporine systemically into the skin to treat psoriasis or other ailments.

### CROSS-REFERENCE TO RELATED APPLICATIONS

This application is a continuation of International Patent Application No. PCT/US2020/067041, filed Dec. 24, 2020 which claims the benefit of U.S. Provisional Patent Application No. 62/953,409, filed on Dec. 24, 2019. Each of the aforementioned patent applications is incorporated herein by reference in its entirety.

### FIELD OF THE INVENTION

The invention relates generally to topical administration of medicaments and more specifically, to methods and formulations for transdermal administration of cyclosporine for treatment of psoriasis.

### BACKGROUND

Psoriasis is an immune-mediated inflammatory disease (IMID) characterized by abnormal patches of skin. It is thought to be a genetic disease that is triggered by environmental factors. The severity of the disease varies from small, localized patches to complete body coverage. There are five main types of psoriasis: plaque, guttate, inverse, pustular, and erythrodermic. Each type of psoriasis manifests differently but they all have overlapping components to the disease. For example, nail manifestations of psoriasis effects 40-45% of people with psoriasis and arthritic manifestations of psoriasis occur in 30% of individuals with the disease of any type. Skin manifestations of psoriasis tend to occur before arthritic manifestations in about 75% of cases.

Psoriasis manifests as an excessive and rapid growth of the outermost layer of the skin (the epidermis). In psoriatic skin, cells are replaced every 3-5 days instead of the normal 28-30 days. This is thought to be caused by premature maturation of keratinocytes due to an inflammatory response in the dermis, which involves certain immune cells (i.e. dendritic cells, macrophages, and T cells). One prominent hypothesis is that psoriasis is caused by a defect in regulatory T cells that induces this cascade of events, ultimately leading to excessive skin cell proliferation.

There are certain genes associated with psoriasis and the results from twin studies indicate that these genetic factors may predispose certain individuals to psoriasis. The activation of the disease can be caused by a variety of factors. In affected individuals, injury to skin can cause psoriasis to occur at the injured spot (Koebner phenomenon). Symptoms are often reported to worsen during winter months and with use of certain medications such as beta-blockers or NSAIDs. Other factors like stress, excessive alcohol consumption, obesity, and chronic infections have also been reported to worsen symptoms.

Plaque psoriasis is the most common type, accounting for 85-90% of cases. Plaque Psoriasis (also known as psoriasis vulgaris), typically manifests as red patches of skin with white scales on top. These patches can occur anywhere on the body but are commonly found on the back of the forearms, shins, navel area, and scalp.

Erythrodermic psoriasis can develop from any of the other types of psoriasis but is often the result of worsening of unstable or untreated plaque psoriasis. A common cause of erythrodermic psoriasis is abrupt cessation of systemic glucocorticoids. This form of the disease occurs when the rash becomes widespread across the body and can be fatal due to the extreme inflammation and exfoliation, disrupting the body's ability to regulate temperature and perform normal skin barrier functions.

The severity of psoriasis is most often determined by the psoriasis area severity index (PASI). PASI assesses the severity of lesions and the area affected and combines these two factors into a single score from 0 (no disease) to 72 (maximal disease). According to this scale, 8% of those affected by psoriasis are categorized as severe.

Psoriasis tends to be more severe if the individual has HIV/AIDS. The rate of psoriatic arthritis is higher in HIV-positive individuals with psoriasis than those that are HIV-negative. If an individual has well-controlled psoriasis, a new HIV infection can trigger a severe flare of psoriasis and/or psoriatic arthritis. In HIV-positive individuals, psoriasis may be so severe that it is untreatable with conventional therapy.

For mild to moderate psoriasis, some topical creams and ointments can be sufficient. Topical corticosteroids are the most frequently prescribed medication for mild to moderate psoriasis. These drugs can reduce inflammation and relieve itching. Other common topicals used for mild to moderate psoriasis include vitamin D analogues (synthetic vitamin D), anthralin, topical retinoids (derived from vitamin A), and topical calcineurin inhibitors. These topicals can alleviate symptoms by slowing the rate of skin cell growth and decreasing inflammation.

Light therapy is another common treatment for mild to moderate psoriasis, often used in tandem with topicals. Exposure to sunlight for UV rays can slow skin cell turnover. UVB phototherapy from artificial light sources is also available. Controlled doses of broadband UVB can be administered to affected areas, often used for mild cases that are resistant to topical treatment. Narrow band UVB can be more effective than broadband UVB and is usually administered a few times per week until symptoms improve. Thereafter, the frequency of treatments can be reduced to once per week.

For cases of severe psoriasis or those resistant to other types of treatment, there are oral or injection drug options, often referred to as systemic treatment. Oral retinoids are more effective than when applied topically and may work for cases with resistance to other treatment. Methotrexate is another oral treatment often prescribed for severe psoriasis that decreases skin cell production and reduces inflammation. There is also a class of drugs that alters the immune system called biologics. These drugs are injected subcutaneously to treat moderate to severe psoriasis.

Topical treatments for mild to moderate psoriasis have their own side effects and vary in efficacy, ultimately being restricted by the severity of the disease on a case-by-case basis. As previously mentioned, topical corticosteroids are the most common treatment for mild psoriasis. However, long-term use or overuse of strong corticosteroids can cause thinning of the skin and topical corticosteroids can stop working over time. For these reasons, use of topical corticosteroids is often recommended as a short-term treatment during flares.

Some vitamin D analogues such as calcipotriene (Dovonex) have the potential to irritate the skin, while others like calcitriol (Vectical) may have less irritation but are much more expensive. Anthralin can also irritate skin and stains most surfaces it touches. Topical retinoids often cause skin irritation and they can also increase sensitivity to sunlight. Additionally, they present a risk of causing birth defects, so they are not recommended to patients that are pregnant, intend to become pregnant, or are breast-feeding. Calcineurin inhibitors can increase the risk of skin cancer or lymphoma and are thus not recommended for long-term use.

Light therapy can be helpful alone or in conjunction with topical treatments, but it has its own issues. Intense or long durations of sun exposure have the potential to worsen symptoms. Broadband UVB can induce redness, itching, and dry skin, while narrow band UVB can cause severe or long-lasting burns. Moreover, light therapy can also increase the risk of skin cancer.

Oral and injected medications for psoriasis are usually reserved for more severe cases due to their side effects. Oral retinoids present a higher risk of severe birth defects than their topical counterparts and should not be used within three years of becoming pregnant. Oral methotrexate can cause upset stomach, loss of appetite, and fatigue. Its more serious side effects include severe liver damage and decreased production of red and white blood cells. Biologics have a significant effect on the immune system and can leave the patient susceptible to life-threatening infections like tuberculosis.

Cyclosporine (cyclosporine A) is an immunosuppressant that can be taken orally or injected. It is used with other medicines to prevent organ rejection by suppressing the immune system. It can also be used to treat rheumatoid arthritis and severe plaque psoriasis when other treatments are ineffective. Cyclosporine can suppress the immune system and slow down the growth of certain immune cells that are involved in the heightened skin production in psoriasis. However, cyclosporine also has many side effects.

Side effects of cyclosporine include kidney problems and high blood pressure, particularly when taken in higher doses and/or long-term therapy. Due to the low bioavailability of oral administration, a higher dose is required for effective treatment of psoriasis. Cyclosporine can also increase the risk of infection and other health problems because it suppresses the immune system.

Attempts at topically administering cyclosporine have been largely unsuccessful. Cyclosporine is a difficult molecule to delivery transdermally due to its high molecular weight and other characteristics. The Lipinski Rule describes molecular properties important for a drug's pharmacokinetics in the human body, including their absorption, distribution, metabolism, and excretion. Based on the criteria of the Lipinski Rule, cyclosporine is unsuitable to conventional topical delivery systems. This is due to its high molecular weight (1202.6 g/mol), its 5 H-bond donors and 12 H-bond acceptors (both of which exceed the Lipinski threshold) and cyclosporine's logP value of 7.5.

Multiple clinical studies attempted to treat psoriasis patients with topical cyclosporine but failed to show any clinical effectiveness in comparison to placebo. The trial treatments included creams with 2% to 5% cyclosporine used over one to two months. Though cyclosporine is effective and transdermal approaches are desired, attempts have thus far been unable to provide a substitute for oral use

Accordingly, an improved method of transdermal administration of cyclosporine is needed. It should overcome the barrier presented by the stratum corneum as well as the deeper layers of skin. Further, it should do so without harsh solvents and present cyclosporine to a localized area with high bioavailability. Aspects of the present invention fulfill these needs and provide further related advantages as described in the following summary.

### SUMMARY OF THE INVENTION

Aspects of the present disclosure teach certain benefits in construction and use which give rise to the exemplary advantages described below.

Embodiments include a transdermal formulation for transdermal administration of cyclosporine. A transdermal delivery formulation can include the following components:
a. cyclosporine at a concentration from 0.5% to 5.0%;
b. isopropyl palmitate at a concentration from 5% to 20%;
c. benzyl alcohol at a concentration of 0.5% to 5%;
d. stearic acid at a concentration from 0.5% to 5%;
e. safflower oil at 1% to 6%;
f. oleic acid at 0.5% to 2%; and
g. deionized water at 20% to 80%;

The transdermal delivery formulation can also include Aveeno® moisturizers, cream, oils, lotions; Jergens® moisturizers, cream, oils, lotions; Honest Company® moisturizers, cream, oils, lotions; Dermologica® moisturizers, cream, oils, lotions; or St. Ives™ moisturizers, cream, oils, lotions.

The transdermal delivery formulation can also include Phospholipon 90G at a concentration of 1% to 20%. In another embodiment, the transdermal delivery formulation includes Durosoft PK-SG at a concentration of 0.5% to 5% and/or Pluronic Gel at a concentration of 5% to 40%.

In another embodiment, the transdermal delivery formulation includes a surfactant, a nonionic detergent and/or a polar gelling agent. The nonionic detergent can lead to a more viscous and cream-like formulation. The polar gelling agent can lead to a more viscous and gel-like formulation.

The cyclosporine concentration can range from 0.05% to 0.1%, from 0.1% to 0.5%, from 0.5% to 2%, from 0.5% to 1.5%, from 1% to 1.5%, from 1% to 2.5%, from 1% to 3%, from 1.5% to 3%, from 1% to 4% or from 1% to 5%. In an embodiment, the cyclosporine concentration is at least 0.5 mg/kg, at least 0.75 mg/kg, at least 1 mg/kg, at least 1.5 mg/kg, at least 2 mg/kg, at least 2.5 mg/kg, at least 3 mg/kg, at least 3.5 mg/kg, at least 4 mg/kg, at least 4.5 mg/kg, at least 5 mg/kg, at least 5.5 mg/kg, at least 6 mg/kg, at least 6.5 mg/kg, at least 7 mg/kg, at least 7.5 mg/kg, at least 8 mg/kg, at least 8.5 mg/kg, at least 9 mg/kg, at least 9.5 mg/kg, at least 10 mb/kg. In an embodiment, the cyclosporine concentration is no more than 0.5 mg/kg, no more than 0.75 mg/kg, no more than 1 mg/kg, no more than 1.5 mg/kg, no more than 2 mg/kg, no more than 2.5 mg/kg, no more than 3 mg/kg, no more than 3.5 mg/kg, no more than 4 mg/kg, no more than 4.5 mg/kg, no more than 5 mg/kg, no more than 5.5 mg/kg, no more than 6 mg/kg, no more than 6.5 mg/kg, no more than 7 mg/kg, no more than 7.5 mg/kg, no more than 8 mg/kg, no more than 8.5 mg/kg, no more than 9 mg/kg, no more than 9.5 mg/kg, no more than 10 mb/kg. In an embodiment, the cyclosporine concentration is about 0.5 mg/kg, about 0.75 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg.

The isopropyl palmitate concentration can range from 1% to 15%, from 2.5% to 15%, from 4% to 15%, from 5% to 10%, from 10% to 15%, from 12% to 15%, from 5% to 8%, from 5% to 15% or from 10% to 20%.

The benzyl alcohol concentration can range from 0.5% to 1.5%, 0.5% to 4%, from 0.75% to 3%, from 1% to 2.5%, from 2% to 4% or from 2.5% to 5%.

The stearic acid concentration can range from 0.5% to 1.5%, from 1.5% to 2.5%, from 3.5% to 5%, from 2% to 5%, from 3% to 5% or from 4% to 5%.

The concentration of safflower oil can range from 1% to 3%, from 1.5% to 2.5%, from 3% to 5% from 4 to 6%, from 4.5% to 6% or from 5% to 6%. The safflower oil can be linoleic acid.

The oleic acid concentration can range from 0.5% to 1%, from 0.5% to 1.5%, from 1% to 1.5% or from 1% to 2%.

The deionized water can range from 20% to 50%, from 25% to 75%, from 30% to 60%, from 40% to 60%, from 40% to 50%, or from 50% to 80%.

Embodiments include a method of administration of cyclosporine to an individual using a transdermal delivery formulation. Embodiments also include a method of treatment of psoriasis using a transdermal delivery formulation. The psoriasis can be plaque psoriasis, guttate psoriasis, inverse psoriasis, pustular psoriasis or erythrodermic psoriasis.

The transdermal delivery formulation can also includes one or more additional agents selected from vitamin D, vitamin D analogues (i.e. synthetic vitamin D), anthralin, topical retinoids (derived from vitamin A), and topical calcineurin inhibitors.

Embodiments also include a method of treatment of a skin condition. The skin condition can be, for example, dermatitis, poison ivy and poison oak, and drug rashes, acne, cold sore, hives, keratosis, rosacea, carbuncle, eczema, cellulitis, atopic dermatitis, Kimura disease, pyoderma gangrenosum, psoriasis, chronic hives, acute systemic mastocytosis, and posterior or intermediate uveitis with noninfective cause

Embodiments also include a method of treating an autoimmune condition. The autoimmune condition can be, for example, lupus erythematosus, rheumatoid arthritis, celiac disease, diabetes mellitus type 1, Graves' disease, inflammatory bowel disease, multiple sclerosis, rheumatoid arthritis, chronic or non-specific inflammation, Guillain-Barre syndrome, chronic inflammatory demyelinating polyneuropathy, Graves' disease, Hashimoto's thyroiditis, myasthenia gravis, vasculitis, fibromyalgia, Crohn's disease, myasthenia gravis, scleritis, vasculitis or systemic lupus erythematosus.

Other features and advantages of aspects of the present invention will become apparent from the following more detailed description, taken in conjunction with the accompanying drawings, which illustrate, by way of example, the principles of aspects of the invention.

### FIGURES

FIGS. 1 and 2. PK data showing resulting mean plasma cyclosporine concentration, measured at 0.5, 1, 2, 4, 8, and 24 hours.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

In an embodiment, a “pharmaceutical composition” is intended to include the combination of an active agent with a carrier, inert or active, in a nonsterile or sterile composition suitable for therapeutic use in vitro, in vivo or ex vivo. In one aspect, the pharmaceutical composition is non-toxic to recipients at the dosage or concentration employed.

In an embodiment, “an effective amount” refers to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effect may, without necessarily being therapeutic, also be a cosmetic effect, in particular for treatment for disorders of the skin described herein.

The term “bioavailability” refers to the fraction of an administered dose of unchanged drug that reaches the systemic circulation. For example, when a medication is administered intravenously, its bioavailability is 100%. However, when a medication is administered via other routes (such as orally), its bioavailability generally decreases due to incomplete absorption and first-pass metabolism. Bioavailability is one of the essential tools in pharmacokinetics, as bioavailability must be considered when calculating dosages for non-intravenous routes of administration.

In an embodiment, “an effective amount” refers, without limitation, to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will, without limitation, vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effect may, without necessarily being therapeutic, also be a cosmetic effect, in particular for treatment for disorders of the skin described herein.

In an embodiment, a “subject” of diagnosis or treatment is, without limitation, a prokaryotic or a eukaryotic cell, a tissue culture, a tissue or an animal, e.g. a mammal, including a human. Non-human animals subject to diagnosis or treatment include, for example, without limitation, a simian, a murine, a canine, a leporid, such as a rabbit, livestock, sport animals, and pets.

In an embodiment, as used herein, the terms “treating,” “treatment” and the like are used herein, without limitation, to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, Pa.—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

As the patients and subjects of the invention method are, in addition to humans, veterinary subjects, formulations suitable for these subjects are also appropriate. Such subjects include livestock and pets as well as sports animals such as horses, greyhounds, and the like.

For purposes herein, a formulation, a formulation for transdermal delivery and a transdermal delivery formulation are each a formulation for transdermal delivery, including, the transdermal delivery of an active ingredient for the treatment of a syndrome and or a disease in an individual.

### Description of Embodiments of the Invention

Transdermal administration refers to applying a substance onto the skin so that it is absorbed into the body for local or systemic distribution. A transdermal solution or transdermal patch is typically placed on one's skin. The solution or patch includes a medicament that is released into the skin. As the layers of skin absorb the solution, the medicament is absorbed via the blood vessels into the bloodstream. From there, the substance can be circulated through the body.

Cyclosporine (also referred to as cyclosporin and cyclosporine A) is a potent immunosuppressant medication. It can be taken by mouth or by injection for treating various conditions related to autoimmunity. For example, cyclosporine is used to treat rheumatoid arthritis, psoriasis, Crohn's disease, nephrotic syndrome, and in organ transplants to prevent rejection. It is believed to work by decreasing the function of lymphocytes.

Embodiments include a transdermal patch, lotion or cream for administration of cyclosporine to a subject. It is placed on the skin to deliver a specific dose of the agent through the skin to a target area. The agent can be delivered across the skin into a localized subdermal location. For example, a cream or lotion can include cyclosporine for treatment of psoriasis. The lotion can also include one or more additional active agents.

There are obvious advantages to transdermal administration of medicaments. It can be applied directly to an affected area as needed. The consumer does not have to schedule and remember to consume doses of pills. Further, transdermal administration is not affected by stomach or digestive issues. Administration across the skin enables drugs to avoid degradation in the gastrointestinal tract or liver. Transdermal delivery is therefore of particular interest for molecules with limited systemic bioavailabilities and short half-lives. Drugs that are absorbed slowly can be more effective. With a transdermal formulation, a medicament can be released in small quantities over a long period of time.

In an alternative embodiment, an agent can be administered using a transdermal or medicated adhesive patch. To release an agent, a patch can utilize a porous membrane covering a reservoir of the agent. Alternatively, the agent can be embedded in layers of the adhesive that release the agent as they dissolve or melt.

An advantage of a transdermal drug delivery route over other types of delivery is that the formulation can provide a controlled release of the agent. Conventional transdermal delivery systems are generally ineffective for use with agents and medications that are large molecules and/or hydrophilic molecules. Further, people can benefit from drugs that are absorbed slowly and regularly. With a transdermal formulation, a medicament can be released in small quantities over a long period of time.

Other advantages are related to dosing. Large doses of agents can cause dose-dependent toxicity in many cases. For example, oral administration of cyclosporine can be harmful to the kidneys. Also, some drugs undergo first-pass metabolism, which prevents their delivery to the desired site of action. Furthermore, many hydrophilic or lipophilic drugs show either poor dissolution or poor absorption on oral administration. With a transdermal formulation, the effective concentration of an agent can be applied at the desired site without painful delivery.

When taken orally or intravenously, cyclosporine can have a range of unwanted side-effects, including convulsions, peptic ulcers, pancreatitis, fever, vomiting, diarrhea, confusion, trouble breathing, high blood pressure, potassium retention (possibly leading to hyperkalemia), kidney and liver dysfunction. With transdermal administration, cyclosporine can be targeted to a particular region of the body. For example, a transdermal cream can be applied directly to regions affected by an autoimmune or skin condition. A patient can apply it directly to knuckles or joints that have inflammation from arthritis. Similarly, the cream can be applied to areas of the skin with dermatitis, hives, keratosis, rosacea or eczema.

Transdermal delivery formulations

In an embodiment, a transdermal delivery formulation containing cyclosporine is comprised of the components of Table 1:

TABLE 1

| Ingredient | Weight (%) |
|---|---|
| Phosphatidylcholine | 7.64% |
| Isopropyl Palmitate | 13.30% |
| Benzyl Alcohol | 1.40% |
| Stearic Acid | 0.62% |
| Safflower Oil | 2.94% |
| Oleic Acid | 0.97% |
| Polyglyceryl-4 laurate | 1.06% |
| Deionized Water | 39.22% |
| Pluronic Gel | 30.85% |
| Cyclosporine A | 2.00% |
| Total | 100.00% |

In an embodiment, a formulation comprises one or more components set forth in Table 1. In a further embodiment, a formulation comprises two, three, four, five, six, seven, eight, nine or ten of the components set forth in Table 1. In an embodiment, lecithin is added as an additional component to the formulation of Table 1.

In an embodiment, lecithin is at a concentration from 5% to 20% of the transdermal formulation. In a further embodiment, the concentration of lecithin can be from 1% to 15%, from 2.5% to 15%, from 4% to 15%, from 5% to 10%, from 10% to 20%, from 15% to 20%, from 5% to 20%, from 8% to 12% or from 1% to 20%. In an embodiment, the concentration of lecithin in a transdermal delivery formulation is at least 2%, at least 5%, at least 10%, at least 15%, at least 20%, at least 25%, at least 28.75%, at least 30%, at least 35%, at least 40% or more. In an aspect, the concentration of lecithin in a transdermal delivery formulation is not more than 10%, not more than 15%, not more than 20%, not more than 25%, not more than 28.75%, not more than 30%, not more than 35%, not more than 40% or more. In an embodiment, the concentration of lecithin in a transdermal delivery formulation is about 10%, about 15%, about 20%, about 25%, at least 28.75%, about 30%, about 35%, about 40% or more. In an aspect, the concentration of lecithin in a transdermal delivery formulation is from 1% to 30%, is from 2.5% to 20%, is from 4% to 15%, is from 5% to 10%, is from 10% to 40%, is from 15% to 35%, is from 20% to 30%, is from 25% to 30%, is from 28% to 29%.

In an embodiment, the concentration of phosphatidylcholine in a transdermal delivery formulation is at least 2%, at least 5%, at least 10%, at least 15%, at least 20%, at least 25%, at least 30%, at least 35%, at least 40% or more.

In an embodiment, the concentration of isopropyl palmitate in a transdermal delivery formulation is at least 2%, at least 5%, at least 10%, at least 15%, at least 20%, at least 25%, at least 28.75%, at least 30%, at least 35%, at least 40% or more.

In an embodiment, the concentration of benzyl alcohol in a transdermal delivery formulation is at least 0.25%, at least 0.5%, at least 0.75%, at least 1%, at least 2%, at least 2.5%, at least 3%, at least 4%, at least 5% or more. In an embodiment, the concentration of benzyl alcohol in a transdermal formulation is about 0.25%, about 0.5%, about 0.75%, about 1%, about 2%, about 2.5%, about 3%, about 4%, about 5% or more. In another embodiment, the concentration of benzyl alcohol in a transdermal formulation is at from 0.25% to 5%; from 0.5% to 4%, from 0.75% to 3%, from 1% to 2.5% or from 0.5% to 2%. In a further embodiment, the concentration of benzyl alcohol in a transdermal formulation is no more than 0.25%, no more than 0.5%, no more than 0.75%, no more than 1%, no more than 2%, no more than 2.5%, no more than 3%, no more than 4%, or no more than 5%.

In another embodiment, the concentration of stearic acid in a transdermal formulation is at least 1%, at least 2%, at least 2.34%, at least 3%, at least 4%, at least 5%, at least 6%, at least 7%, at least 8%, at least 9%, at least 10% or more. In another aspect, the concentration of stearic acid in a transdermal formulation is no more than 1%, no more than 2%, no more than 2.34%, no more than 3%, no more than 4%, no more than 5%, no more than 6%, no more than 7%, no more than 8%, no more than 9%, no more than 10% or more. In another aspect, the concentration of stearic acid in a transdermal formulation is about 1%, about 2%, about 2.34%, about 3%, about 4%, about 5%, about 6%, about 7%, about 8%, about 9%, about 10% or more. In another aspect, the concentration of stearic acid in a transdermal formulation is from 1% to 10%, from 2% to 9%, from 2% to 3%, from 2.34% to 2.5%, from 3% to 8%, from 4% to 7%, from 5% to 6%, from 0.2% to 10%, from 0.2% to 7% from 0.2% to 5%, from 0.2% to 3%, from 1% to 8%, from 3% to 7%, from 4% to 6%, from 2% to 7%, or from 1.5% to 2.5%.

In an embodiment, the concentration of safflower (Carthamus tinctorius) oil, including a linoleic acid, in a transdermal delivery formulation is at least 1%, at least 5%, at least 7.5%, at least 10%, at least 11%, at least 11.06%, at least 12%, at least 13%, at least 14%, at least 15%, at least 16%, at least 17%, at least 18%, at least 19%, at least 20% or more. In an aspect, the concentration of safflower oil in a transdermal delivery formulation is about 1%, about 5%, about 7.5%, about 10%, about 11%, about 11.06%, about 12%, about 13%, about 14%, about 15%, about 16%, about 17%, about 18%, about 19%, about 20% or more. In an embodiment, the concentration of safflower oil in a transdermal delivery formulation is from 1% to 20%, from 1% to 10%, from 1% to 15%, from 2% to 10% from 5 to 10%, from 1% to 7%, from 1% to 5% from 2% to 4%, from 5% to 19%, from 7.5% to 18%, from 10% to 17%, from 11% to 16%, from 11.06% to 12%, from 11% to 12%, from 12% to 14%, from 13% to 14%, from 10% to 12%, from 10.5% to 12.5% or from 11% to 11.25%. In an embodiment, the concentration of safflower oil in a transdermal delivery formulation is no more than 1%, no more than 5%, no more than 7.5%, no more than 10%, no more than 11%, no more than 11.06%, no more than 12%, no more than 13%, no more than 14%, no more than 15%, no more than 16%, no more than 17%, no more than 18%, no more than 19%, no more than 20%, no more than 25% or more.

In a further embodiment, the concentration of oleic acid in a transdermal delivery formulation is at least 1%, at least 2%, at least 3%, at least 3.65%, at least 4%, at least 5%, at least 6%, at least 7%, at least 8%, at least 9%, at least 10% or more. In a further embodiment, the concentration of oleic acid in a transdermal delivery formulation is about 1%, about 2%, about 3%, about 3.5%, about 4%, about 5%, about 6%, about 7%, about 8%, about 9%, about 10% or more. In a further aspect, the concentration of oleic acid in a transdermal delivery formulation is no more than 1%, no more than 2%, no more than 3%, no more than 3.5%, no more than 4%, no more than 5%, no more than 6%, no more than 7%, no more than 8%, no more than 9%, no more than 10% or more. In another embodiment, the concentration of oleic acid in a transdermal formulation is from 1% to 10%, from 2% to 9%, from 2% to 3%, from 3% to 4%, from 3% to 8%, from 4% to 7%, from 5% to 6%, from 2 to 2.5%, from 0.2% to 10%, from 0.2% to 7.5% from 0.2% to 5%, from 1% to 7.5%, from 2 to 5%, from 3% to 5%, or from 2.5% to 4%.

In an embodiment, the concentration of Polyglyceryl-4 laurate is from at least 1%, at least 2%, at least 3%, at least 4%, at least 5% or from 0.5% to 5%.

In an embodiment, the concentration of deionized water in a transdermal formulation is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 65%, at least 70%, at least 75 or more. In an embodiment, the concentration of deionized water in a transdermal formulation is about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 65%, about 70%, about 75% or more. In an embodiment, the concentration of deionized water in a transdermal formulation is from 0.1% to 5%, from 0.2% to 4%, from 0.3% to 3%, from 0.4% to 2%, from 0.5% to 1%, from 0.6% to 0.9%, from 0.7% to 0.8%, from 0.4% to 1.5%, from 0.3% to 0.7%, from 1% to 50%, from 10% to 40% from 10% to 45%, from 10% to 30%, from 10% to 20%, from 20% to 50%, from 20% to 45%, from 20% to 40%, from 20% to 30z% from 5% to 30%, from 5% to 25%, from 5% to 20%, from 5% to 15%, or from 0.4% to 0.6%. In an embodiment, the concentration of deionized water in a transdermal formulation is no more than 0.1%, no more than 0.2%, no more than 0.3%, no more than 0.4%, no more than 0.5%, no more than 0.6%, no more than 0.7%, no more than 0.8%, no more than 0.9%, no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, no more than 50%, no more than 51%, no more than 52%, no more than 53%, no more than 54%, no more than 55%, no more than 56%, no more than 57%, no more than 58%, no more than 59%, no more than 60%, no more than 65%, no more than 70%, no more than 75% or more.

The pluronic gel can have a concentration of at least 2%, at least 5%, at least 10%, at least 15%, at least 20%, at least 25%, at least 30%, at least 35%, at least 40% or more.

In an embodiment, the concentration of cyclosporine in a transdermal delivery formulation is at least 0.25%, at least 0.5%, at least 0.75%, at least 1%, at least 1.5%, at least 2%, at least 2.5%, at least 3%, at least 3.5%, at least 4%, at least 4.5%, at least 5%, at least 6%, at least 7%, at least 8%, at least 10% or more. In an embodiment, the cyclosporine concentration is no more than 0.5%, no more than 0.75%, no more than 1%, no more than 1.5%, no more than 2%, no more than 2.5%, no more than 3%, no more than 3.5%, no more than 4%, no more than 4.5%, no more than 5%, no more than 5.5%, no more than 6%, no more than 6.5%, no more than 7%, no more than 7.5%, no more than 8%, no more than 8.5%, no more than 9%, no more than 9.5%, no more than 10%. In an embodiment, the cyclosporine concentration is about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%.

In an embodiment, the concentration of cyclosporine in a transdermal formulation is 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, 205 mg/kg, 210 mg/kg, 215 mg/kg, 220 mg/kg, 225 mg/kg, 230 mg/kg, 235 mg/kg, 240 mg/kg, 245 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg or greater than 500 mg/kg.

In an embodiment, the concentration of cyclosporine in a transdermal formulation is from 0.1% to 10%, from 0.5% to 8%, from 1% to 7%, from 1.5% to 6%, from 2% to 5%, from 2.5% to 4%, from 2% to 4%, from 1.5% to 4%, from 1.5% to 5%, from 2% to 6%, from 2% to 3%, from 2.25% to 2.75% or from 2.4% to 2.6%. In an embodiment, the cyclosporine concentration is at least 0.5%, at least 0.75%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%. In an embodiment, the cyclosporine concentration is no more than 0.5%, no more than 0.75%, no more than 1%, no more than 1.5%, no more than 2%, no more than 2.5%, no more than 3%, no more than 3.5%, no more than 4%, no more than 4.5%, no more than 5%, no more than 5.5%, no more than 6%, no more than 6.5%, no more than 7%, no more than 7.5%, no more than 8%, no more than 8.5%, no more than 9%, no more than 9.5%, no more than 10%. In an embodiment, the cyclosporine concentration is about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%.

In an embodiment, the concentration of cyclosporine in a transdermal formulation is at least 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, 205 mg/kg, 210 mg/kg, 215 mg/kg, 220 mg/kg, 225 mg/kg, 230 mg/kg, 235 mg/kg, 240 mg/kg, 245 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg or greater than 500 mg/kg. In an embodiment, the cyclosporine concentration is at least 0.5 mg/kg, at least 0.75 mg/kg, at least 1 mg/kg, at least 1.5 mg/kg, at least 2 mg/kg, at least 2.5 mg/kg, at least 3 mg/kg, at least 3.5 mg/kg, at least 4 mg/kg, at least 4.5 mg/kg, at least 5 mg/kg, at least 5.5 mg/kg, at least 6 mg/kg, at least 6.5 mg/kg, at least 7 mg/kg, at least 7.5 mg/kg, at least 8 mg/kg, at least 8.5 mg/kg, at least 9 mg/kg, at least 9.5 mg/kg, at least 10 mg/kg. In an embodiment, the cyclosporine concentration is no more than 0.5 mg/kg, no more than 0.75 mg/kg, no more than 1 mg/kg, no more than 1.5 mg/kg, no more than 2 mg/kg, no more than 2.5 mg/kg, no more than 3 mg/kg, no more than 3.5 mg/kg, no more than 4 mg/kg, no more than 4.5 mg/kg, no more than 5 mg/kg, no more than 5.5 mg/kg, no more than 6 mg/kg, no more than 6.5 mg/kg, no more than 7 mg/kg, no more than 7.5 mg/kg, no more than 8 mg/kg, no more than 8.5 mg/kg, no more than 9 mg/kg, no more than 9.5 mg/kg, no more than 10 mg/kg. In an embodiment, the cyclosporine concentration is about 0.5 mg/kg, about 0.75 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg.

In an embodiment, the concentration of cyclosporine in a transdermal formulation is about 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, 205 mg/kg, 210 mg/kg, 215 mg/kg, 220 mg/kg, 225 mg/kg, 230 mg/kg, 235 mg/kg, 240 mg/kg, 245 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg or greater than 500 mg/kg . In an embodiment, the cyclosporine concentration is at least 0.5 mg/kg, at least 0.75 mg/kg, at least 1 mg/kg, at least 1.5 mg/kg, at least 2 mg/kg, at least 2.5 mg/kg, at least 3 mg/kg, at least 3.5 mg/kg, at least 4 mg/kg, at least 4.5 mg/kg, at least 5 mg/kg, at least 5.5 mg/kg, at least 6 mg/kg, at least 6.5 mg/kg, at least 7 mg/kg, at least 7.5 mg/kg, at least 8 mg/kg, at least 8.5 mg/kg, at least 9 mg/kg, at least 9.5 mg/kg, at least 10 mg/kg. In an embodiment, the cyclosporine concentration is no more than 0.5 mg/kg, no more than 0.75 mg/kg, no more than 1 mg/kg, no more than 1.5 mg/kg, no more than 2 mg/kg, no more than 2.5 mg/kg, no more than 3 mg/kg, no more than 3.5 mg/kg, no more than 4 mg/kg, no more than 4.5 mg/kg, no more than 5 mg/kg, no more than 5.5 mg/kg, no more than 6 mg/kg, no more than 6.5 mg/kg, no more than 7 mg/kg, no more than 7.5 mg/kg, no more than 8 mg/kg, no more than 8.5 mg/kg, no more than 9 mg/kg, no more than 9.5 mg/kg, no more than 10 mg/kg. In an embodiment, the cyclosporine concentration is about 0.5 mg/kg, about 0.75 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg.

In an embodiment, the concentration of cyclosporine in a transdermal formulation is no more than 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, 205 mg/kg, 210 mg/kg, 215 mg/kg, 220 mg/kg, 225 mg/kg, 230 mg/kg, 235 mg/kg, 240 mg/kg, 245 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg or greater than 500 mg/kg. In an embodiment, the cyclosporine concentration is at least 0.5 mg/kg, at least 0.75 mg/kg, at least 1 mg/kg, at least 1.5 mg/kg, at least 2 mg/kg, at least 2.5 mg/kg, at least 3 mg/kg, at least 3.5 mg/kg, at least 4 mg/kg, at least 4.5 mg/kg, at least 5 mg/kg, at least 5.5 mg/kg, at least 6 mg/kg, at least 6.5 mg/kg, at least 7 mg/kg, at least 7.5 mg/kg, at least 8 mg/kg, at least 8.5 mg/kg, at least 9 mg/kg, at least 9.5 mg/kg, at least 10 mg/kg. In an embodiment, the cyclosporine concentration is no more than 0.5 mg/kg, no more than 0.75 mg/kg, no more than 1 mg/kg, no more than 1.5 mg/kg, no more than 2 mg/kg, no more than 2.5 mg/kg, no more than 3 mg/kg, no more than 3.5 mg/kg, no more than 4 mg/kg, no more than 4.5 mg/kg, no more than 5 mg/kg, no more than 5.5 mg/kg, no more than 6 mg/kg, no more than 6.5 mg/kg, no more than 7 mg/kg, no more than 7.5 mg/kg, no more than 8 mg/kg, no more than 8.5 mg/kg, no more than 9 mg/kg, no more than 9.5 mg/kg, no more than 10 mg/kg. In an embodiment, the cyclosporine concentration is about 0.5 mg/kg, about 0.75 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg.

In certain embodiments cyclosporine is administered topically or transdermally such that the dose results in a subject intake of at least about 0.1 nmol/hr/kg, at least about 0.5 nmol/hr/kg, at least about 0.7 nmol/hr/kg, at least about 1.0 nmol/hr/kg, at least about 1.1 nmol/hr/kg, at least about 1.2 nmol/hr/kg, at least about 1.3 nmol/hr/kg, at least about 1.4 nmol/hr/kg, at least about 1.5 nmol/hr/kg, at least about 1.6 nmol/hr/kg, at least about 1.7 nmol/hr/kg, at least about 1.8 nmol/hr/kg, at least about 1.9 nmol/hr/kg, at least about 2.0 nmol/hr/kg, at least about 2.5 nmol/hr/kg, at least about 3.0 nmol/hr/kg, at least about 3.5 nmol/hr/kg, at least about 4.0 nmol/hr/kg, at least about 5 nmol/hr/kg, at least about 10 nmol/hr/kg, at least about 25 nmol/hr/kg, at least about 50 nmol/hr/kg, at least about 100 nmol/hr/kg, at least about 500 nmol/hr/kg, or at least about 1 μmol/hr/kg.

In certain embodiments cyclosporine is administered topically or transdermally such that the dose results in a peak plasma concentration of cyclosporine ranging from about 1 μg/ml to 50 μg/ml, about 5 μg/ml to about 45 μg/ml, about 5 μg/ml to about 40 μg/ml, about 5 μg/ml to about 35 μg/ml, about 5 μg/ml to about 30 μg/ml, about 5 μg/ml to about 25 μg/ml, about 1 μg/ml to about 45 μg/ml, about 1 μg/ml to about 40 μg/ml, about 1 μg/ml to about 35 μg/ml, about 1 μg/ml to about 30 μg/ml, about 1 μg/ml to about 25 μg/ml, about 1 μg/ml to about 20 μg/ml, about 1 μg/ml to about 15 μg/ml, about 1 μg/ml to about 10 μg/ml, about 1 μg/ml to about 9 μg/ml, about 1 μg/ml to about 8 μg/ml, about 1 μg/ml to about 7 μg/ml, about 1 μg/ml to about 6 μg/ml, and about 1 μg/ml to about 5 μg/ml.

In certain embodiments cyclosporine is administered topically or transdermally so that plasma cyclosporine concentration ranges from about 1 ng/ml to 5 ng/ml, about 1 ng/ml to 10 ng/ml, about 5 ng/ml to 10 ng/ml, about 5 ng/ml to 20 ng/ml, about 10 ng/ml to 20 ng/ml, about 20 ng/ml to 40 ng/ml, about 10 ng/ml to 50 ng/ml, about 20 ng/ml to 80 ng/ml, about 1 ng/ml to 500 μg/ml, about 10 ng/ml to 500 μg/ml, about 100 ng/ml to 500 μg/ml, about 1 μg/ml to 500 μg/ml, about 10 μg/ml to 500 μg/ml, about 25 μg/ml to 500 μg/ml, about 25 μg/ml to about 450 μg/ml, about 25 μg/ml to about 400 μg/ml, about 25 μg/ml to about 350 μg/ml, about 25 μg/ml to about 300 μg/ml or about 25 μg/ml to about 250 μg/ml.

In further embodiments, a cyclosporine is administered topically or transdermally so that plasma concentration is at least 1 ng/ml, at least 5 ng/ml, at least 10 ng/ml, at least 15 ng/ml, at least 20 ng/ml, at least 25 ng/ml, at least 50 ng/ml, at least 100 ng/ml, at least 250 ng/ml, at least 0.5 μg/ml, at least 0.75 μg/ml, at least 1 μg/ml, at least 2 μg/ml, at least 3 μg/ml, at least 4 μg/ml, at least 5 μg/ml, at least 6 μg/ml, at least 7 μg/ml, at least 8 μg/ml, at least 9 μg/ml, at least 10 μg/ml, at least 15 μg/ml, at least 20 μg/ml, at least 25 μg/ml, at least 30 μg/ml, at least 35 μg/ml, at least 40 μg/ml, at least 45 μg/ml, at least 50 μg/ml, at least 55 μg/ml, at least 60 μg/ml, at least 65 μg/ml, at least 70 μg/ml, at least 75 μg/ml, at least 80 μg/ml, at least 85 μg/ml, at least 90 μg/ml, at least 95 μg/ml, at least 100 μg/ml or more than 100 μg/ml.

The present disclosure herein demonstrates transdermal delivery of an agent without many of the negative effects on color, smell, grittiness and stability driven by the use of lecithin organogel. Moreover, the methods describe herein improve transdermal penetration.

In an embodiment, a transdermal delivery formulation contains a phosphatide in a concentration of at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

Phosphatides—soy lecithin contains about 57.5% w/w phosphatides. The primary phosphatides found in soy lecithin are inositol phosphatides (20.5% w/w of soy lecithin), phosphatidylcholine (20%), and phosphatidylethanolamine (11% w/w of soy lecithin). In some embodiments, phosphatidylcholine is used for the full amount (57.5% w/w of soy lecithin) as it is known to aide in skin penetration. Other phosphatides include phosphatidic acid, phosphatidylserine and phosphatidylinositol.

In an embodiment, a transdermal delivery formulation contains a sterol or benzyl alcohol in a concentration of at least 1%, at least 2%, at least 3%, at least 4%, at least 5%, at least 6%, at least 7%, at least 8%, at least 9%, at least 10%, at least 15%, at least 20%, at least 25%, at least 30% or more w/w of the transdermal delivery formulation.

Sterols—soy lecithin contains about 2.5% w/w sterols. In some embodiments, benzyl alcohol is used in substitution of the sterol in a transdermal delivery formulation to act as a penetration enhancer. In another embodiment, a sterol is cholesterol, ergosterol, hopanoids, hydroxysteroid, phytosterol and/or other steroids.

In an embodiment, a transdermal delivery formulation contains a carbohydrate in a concentration of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

Carbohydrates—Soy lecithin contains about 5% w/w free carbohydrates. In some embodiments, glucose is used in substitution of a free carbohydrate to maintain the ratio of sugars in the transdermal delivery formulation disclosed herein. In another embodiment, a carbohydrate is a monosaccharide, a disaccharide, a polyol, a malto-oligosaccharide, an oligosaccharide, a starch, a polysaccharide. In a further embodiment, a carbohydrate is glucose, galactose, fructose, xylose, sucrose, lactose, maltose, trehalose, sorbitol, mannitol, maltodextrins, raffinose, stachyose, fructo-oligosaccharide, amylose, amylopectin, modified starches, glycogen, cellulose, hemicellulose, pectin and/or hydrocolloid.

Moisture—In some embodiments, the transdermal delivery formulation maintains the about 1% w/w of water contained in soy lecithin.

In an embodiment, a transdermal delivery formulation contains water in a concentration of at least 0.1%, at least 0.2%, at least 0.3%, at least 0.4%, at least 0.5%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

Fatty acids—Soy lecithin contains about 34% w/w fatty acids, including 18-19% w/w linoleic acid, 1-2% w/w alpha-linoleic acid, 8-9% w/w oleic acid, about 5% w/w palmitic acid, and 1-2% w/w stearic acids. In some embodiments, the fatty acids are similar to the fatty acids contained in soy lecithin. In an embodiment, alpha-linoleic is removed from the transdermal delivery formulation as it is known to oxidize and can become rancid. In some embodiments, the amount of stearic acid has been increased (i.e., enhancing stability of the formulation) or linoleic acid (i.e., enhances skin penetration). In some embodiments, a seed oil such as purified safflower oil is used in a transdermal delivery formulation due to its similarity to the fatty acids found in soy lecithin, its relative availability and its low cost. In some embodiments, the fatty acid content of a transdermal formulation can be adjusted with a different seed oil through the addition of smaller amounts of the fatty acids disclosed herein.

In a further embodiment, a fatty acid is a saturated or an unsaturated fatty acid. In another embodiment, an unsaturated fatty acid is myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and/or docosahexaenoic acid. In an embodiment, a saturated fatty acid is caprylic acid, capric acid, lauric acid, myristic acid, palmitic acid, stearic acid, arachidic acid, behenic acid, lignoceric acid and/or cerotic acid. In another embodiment, the fatty acid is a dietary fat and include duct fat, lard, tallow, butter, coconut oil, cocoa butter, palm kernel oil, palm oil, cottonseed oil, wheat germ oil, soybean oil, olive oil, corn oil, sunflower oil, safflower oil, hemp oil and/or canola/rapeseed oil.

In some embodiments, carotenoids are excluded from the formulations disclosed. Herein, we describe formulations demonstrating the replacement of lecithin organogel (i.e., lecithin and a solvent like isopropyl palmitate).

In an embodiment, the concentration of phosphatidylcholine in a transdermal delivery formulation is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, at least 10%, at least 15%, at least 20%, at least 25%, at least 28.75%, at least 30%, at least 35%, at least 40% or more. In an embodiment, the concentration of phosphatidylcholine in a transdermal delivery formulation is not more than 0.1%, not more than 0.2%, not more than 0.3%, not more than 0.4%, not more than 0.5%, not more than 0.6%, not more than 0.7%, not more than 0.8%, not more than 0.9%, not more than 1%, not more than 2%, not more than 3%, not more than 4%, not more than 5%, not more than 6%, not more than 7%, not more than 8%, not more than 9%, not more than 10%, not more than 15%, not more than 20%, not more than 25%, not more than 28.75%, not more than 30%, not more than 35%, not more than 40% or more. In an aspect, the concentration of phosphatidylcholine in a transdermal delivery formulation is about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 15%, about 20%, about 25%, about 28.75%, about 30%, about 35%, about 40% or more. In an embodiment, the concentration of phosphatidylcholine in a transdermal delivery formulation is from 10% to 40%, is from 15% to 35%, is from 20% to 30%, is from 25% to 30%, is from 28% to 29%.

In another embodiment, the concentration of a carbohydrate in a transdermal delivery formulation is at least 0.1%, at least 0.2%, at least 0.3%, at least 0.4%, at least 0.5%, at least 0.6%, at least 0.7%, at least 0.8%, at least 0.9%, at least 1%, at least 2%, at least 2.5%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17 18%, at least 19%, at least 20% or more. In another aspect, the concentration of a carbohydrate in a transdermal delivery formulation is about 0.1%, about 0.2%, about 0.3%, about 0.4%, about 0.5%, about 0.6%, about 0.7%, about 0.8%, about 0.9%, about 1%, about 2%, about 2.5%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% or more. In another embodiment, the concentration of a carbohydrate in a transdermal delivery formulation is no more than 0.1%, no more than 0.2%, no more than 0.3%, no more than 0.4%, no more than 0.5%, no more than 0.6%, no more than 0.7%, no more than 0.8%, no more than 0.9%, no more than 1%, no more than 2%, no more than 2.5%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20% or more. In another aspect, the concentration of a carbohydrate in a transdermal delivery formulation is from 1% to 10%, is from 2% to 9%, is from 2.5% to 5%, is from 2% to 3%, is from 3% to 8%, if from 4% to 7%, if from 5% to 6%, is from 2% to 4%, is from 1.5% to 3.5, is from 0.1% to 3%, is from 0.5% to 5%, is from 0.5% to 3%, if from 0.5% to 2%, is from 0.5% to 7%.

In an aspect, the concentration of safflower oil in a transdermal delivery formulation is at least 1%, at least 5%, at least 7.5%, at least 10%, at least 11%, at least 11.06%, at least 12%, at least 13%, at least 14%, at least 15%, at least 16%, at least 17%, at least 18%, at least 19%, at least 20% or more. In an aspect, the concentration of safflower oil in a transdermal delivery formulation is about 1%, about 5%, about 7.5%, about 10%, about 11%, about 11.06%, about 12%, about 13%, about 14%, about 15%, about 16%, about 17%, about 18%, about 19%, about 20% or more. In an aspect, the concentration of safflower oil in a transdermal delivery formulation is from 1% to 20%, from 5% to 19%, from 7.5% to 18%, from 10% to 17%, from 11% to 16%, from 11.06%, 12% from 11% to 12%, from 12% to 14%, from 13% to 14%, from 10% to 12%, from 10.5% to 12.5% or from 11% to 11.25%. In an aspect, the concentration of safflower oil in a transdermal delivery formulation is no more than 1%, no more than 5%, no more than 7.5%, no more than 10%, no more than 11%, no more than 11.06%, no more than 12%, no more than 13%, no more than 14%, no more than 15%, no more than 16%, no more than 17%, no more than 18%, no more than 19%, or no more than 20%.

In an aspect, the concentration of isopropyl palmitate in a transdermal formulation is at least 10%, at least 20%, at least 25%, at least 30%, at least 35%, at least 40%, at least 45%, at least 50%, at least 55%, at least 60%, at least 65%, at least 70%, at least 75% or more. In an aspect, the concentration of isopropyl palmitate in a transdermal formulation is about 10%, about 20%, about 25%, about 30%, about 40%, about 45%, about 50%, about 55%, about 60%, about 65%, about 70%, about 75% or more. In an aspect, the concentration of isopropyl palmitate in a transdermal formulation is no more than 10%, no more than 20%, no more than 25%, no more than 30%, no more than 40%, no more than 45%, no more than 50%, no more than 55%, no more than 60%, no more than 65%, no more than 70%, no more than 75% or more. In an aspect, the concentration of isopropyl palmitate in a transdermal formulation is from 10% to 75%, from 20% to 70%, from 25% to 65%, from 30% to 60%, from 40% to 55%, from 45% to 50%, from 40% to 60%, from 45% to 55% or from 47% to 53%.

Certain components or ingredients of a transdermal delivery formulation provided herein may be supplemented with components described in greater detail in the inventor's related applications mentioned above, including U.S. application Ser. No. 16/132,358 filed Sep. 14, 2018, entitled ‘Methods and Formulations For Transdermal Administration Of Buffering Agents’, International Patent Application No. PCT/US18/51250 filed Sep. 14, 2018, entitled ‘Methods of Administration and Treatment’, and International Patent Application PCT/US18/28017 by Bruce Sand filed Apr. 17, 2018, entitled ‘Parental non-systemic administration of buffering agents for inhibiting metastasis of solid tumors, hyperpigmentation and gout’, all incorporated by reference in their entirety herein.

A transdermal delivery formulation comprises of mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize one or more distinct permeation enhancers.

In some embodiments, the cyclosporine is formulated with Aveeno® moisturizers, cream, oils, lotions; Jergens® moisturizers, cream, oils, lotions; Honest Company® moisturizers, cream, oils, lotions; Dermologica® moisturizers, cream, oils, lotions; or St. Ives™ moisturizers, cream, oils, lotions.

The transdermal delivery formulation is a multi-component mixture, whereby the particular concentrations of the penetration enhancers are informed in part by the particle size of the cyclosporine component. The formulation enables the cyclosporine component to become bio-available to the target site within minutes of topical administration. In some embodiments, the transdermal delivery formulation comprises an alcohol in an amount less than 5% w/w of the formulation.

For topical administration, and in particular transdermal administration, a transdermal delivery formulation will comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. In some embodiments, suitable penetrants include those that are described in the above-referenced US2009/0053290 ('290), WO2014/209910 ('910), and WO2017/127834. In addition to transdermal delivery formulations with penetrants, transdermal delivery can be effected by mechanically disrupting the surface of the skin to encourage penetration, or simply by supplying the formulation applied to the skin under an occlusive patch.

Alternatively, the transdermal delivery formulation comprises a completion component as well as one or more electrolytes sufficient to impart viscosity and viscoelasticity, one or more surfactants and an alcohol. The completion component can be a polar liquid, a non-polar liquid or an amphiphilic substance. The penetrant may further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

Suitable gelling components also include isopropyl palmitate, ethyl laurate, ethyl myristate and isopropyl myristate. In some embodiments, a transdermal delivery formulation comprises a gelling agent in an amount less than 1% w/w, less than 2% w/w, less than 3% w/w, less than 4% w/w, less than 5% w/w, less than 6% w/w, less than 7% w/w, less than 8% w/w, less than 9% w/w, less than 10% w/w, less than 11% w/w, less than 12% w/w, less than 13% w/w, less than 14% w/w, less than 15% w/w, less than 16% w/w, less than 17% w/w, less than 18% w/w, less than 19% w/w, less than 20% w/w, less than 25% of a transdermal delivery formulation. Certain hydrocarbons, such as cyclopentane, cyclooctane, trans-decalin, trans-pinane, n-pentane, n-hexane, n-hexadecane may also be used. In some embodiments, the transdermal delivery formulation comprises a mixture of xanthan gum, sclerotium gum, pullulan, or a combination thereof in an amount less than 1% w/w, less than 2% w/w, less than 3% w/w, less than 4% w/w, less than 5% w/w, less than 6% w/w, less than 7% w/w, less than 8% w/w, less than 9% w/w, less than 10% w/w, less than 11% w/w, less than 12% w/w, less than 13% w/w, less than 14% w/w, less than 15% w/w, less than 16% w/w, less than 17% w/w, less than 18% w/w, less than 19% w/w, less than 20% w/w, less than 25% of the formulation. In some embodiments, a transdermal delivery formulation comprises Siligel™ in an amount between about 1-5% w/w or 5-15% w/w, or an equivalent mixture of xanthan gum, sclerotium gum, and pullulan. In some embodiments, a transdermal delivery formulation comprises a mixture of caprylic triglycerides, medium chain triglycerides (MSTs) and capric triglycerides in amount less than 1% w/w, less than 2% w/w, less than 3% w/w, less than 4% w/w, less than 5% w/w, less than 6% w/w, less than 7% w/w, less than 8% w/w, less than 9% w/w, less than 10% w/w, less than 11% w/w, less than 12% w/w, less than 13% w/w, less than 14% w/w, less than 15% w/w, less than 16% w/w, less than 17% w/w, less than 18% w/w, less than 19% w/w, less than 20% w/w, less than 25% of the formulation. In some embodiments, a transdermal delivery formulation comprises Myritol® 312 in an amount between about 0.5-10% w/w or less than 1% w/w, less than 2% w/w, less than 3% w/w, less than 4% w/w, less than 5% w/w, less than 6% w/w, less than 7% w/w, less than 8% w/w, less than 9% w/w, less than 10% w/w, less than 11% w/w, less than 12% w/w, less than 13% w/w, less than 14% w/w, less than 15% w/w, less than 16% w/w, less than 17% w/w, less than 18% w/w, less than 19% w/w, less than 20% w/w, less than 25%, or an equivalent mixture of caprylic triglycerides, MCTs and capric triglycerides.

In some embodiments, a transdermal delivery formulation comprises phosphatidylcholine in an amount between about 10-90% w/w or 10-50% w/w of the formulation or at least 10% w/w, at least 20% w/w, at least 30% w/w, at least 40% w/w, at least 50% w/w, at least 60% w/w, at least 70% w/w, at least 80% w/w, at least 90% w/w or at least 95% w/w. In some embodiments, a transdermal delivery formulation comprises phosphatidylcholine in amount less than 7% w/w, less than 8% w/w, less than 9% w/w, less than 10% w/w, less than 11% w/w, less than 12% w/w, less than 13% w/w, less than 14% w/w, less than 15% w/w, less than 16% w/w, less than 17% w/w or less than 18% w/w of the formulation. In some embodiments, a transdermal delivery formulation comprises a phospholipid in amount less than 20% w/w, less than 30% w/w, less than 40% w/w, less than or 50% w/w of the formulation. In some embodiments, a transdermal delivery formulation comprises a mixture of tridecane and undecane in amount less than 2% w/w, 3% w/w, 4% w/w, 5% w/w, 6% w/w, 7% w/w, or 8% w/w of the formulation. In some embodiments, the formulation comprises Cetiol Ultimate® in an amount less than about 2% w/w, 3% w/w, 4% w/w, 5% w/w, 6% w/w, 7% w/w, 8% w/w, 9% w/w, or 10% w/w, or an equivalent mixture of tridecane and undecane. In some embodiments, a transdermal delivery formulation comprises cetyl alcohol in amount less than 2% w/w, 3% w/w, 4% w/w, 5% w/w, 6% w/w, 7% w/w, 8% w/w, 9% w/w, or 10% w/w of the formulation. In some embodiments, the formulation comprises benzyl alcohol in an amount less than about 2% w/w, 3% w/w, 4% w/w, 5% w/w, 6% w/w, 7% w/w, 8% w/w, 9% w/w, or 10% w/w. In some embodiments, a transdermal delivery formulation comprises stearic acid in an amount less than 2% w/w, 3% w/w, 4% w/w, 5% w/w, 6% w/w, 7% w/w, 8% w/w, 9% w/w, or 10% w/w of the formulation. In some embodiments, the transdermal delivery formulation comprises phosphatidylcholine, hydrogenated phosphatidylcholine, phosphatidylserine, phosphatidylethanolamine, phosphatidylinositol, one or more phosphatides, one or more inositol phosphatides, or combinations thereof, in amount less than 30% w/w or in amount less than 12% w/w of the formulation.

An additional component in a transdermal delivery formulation of the disclosure is an alcohol. Benzyl alcohol and/or ethanol are illustrated in the examples. In particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 3% w/w, 4% w/w, 5% w/w, 6% w/w, 7% w/w, 8% w/w, 9% w/w, or 10% w/w, and other intermediate weight percentages are included. Due to the aromatic group present in a transdermal delivery formulation such as benzyl alcohol, the molecule has a polar end (the alcohol end) and a non-polar end (the benzene end). This enables the agent to dissolve a wider variety of transdermal delivery formulation components.

In some embodiments, as noted above, the performance of a transdermal delivery formulation is further improved by including a nonionic detergent and polar gelling agent or including a powdered surfactant. In both aqueous and anhydrous forms of the composition, detergents, typically nonionic detergents are added. In general, the nonionic detergent should be present in an amount between about 1% w/w to 30% w/w of a transdermal delivery formulation. Typically, in the compositions wherein a transdermal delivery formulation is topped off with a polar or aqueous solution containing detergent, the amount of detergent is relatively low—e.g., 2-25% w/w, or 5-15% w/w or 7-12% w/w of a transdermal delivery formulation. However, in compositions that are essentially anhydrous and are topped-off by powdered detergent, relatively higher percentages are usually used—e.g., 20-60% w/w.

In some embodiments, a transdermal delivery formulation further comprises a detergent portion in an amount between about 1 to 70% w/w or 1 to 60% w/w of a transdermal delivery formulation. In some embodiments, the nonionic detergent provides suitable handling properties whereby the formulations are gel-like or creams at room temperature. To exert this effect, the detergent, typically a poloxamer, is present in an amount between about 2-12% w/w of a transdermal delivery formulation, preferably between about 5-25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as the non-ionic surfactant Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, a transdermal delivery formulation also contains surfactant, typically, nonionic surfactant at 2-25% w/w of a transdermal delivery formulation along with a polar solvent wherein the polar solvent is present in an amount at least in molar excess of the nonionic surfactant. In these embodiments, typically, the composition comprises the above-referenced amounts of a transdermal delivery formulation and benzyl alcohol along with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like. In some embodiments, the detergent portion comprises a nonionic surfactant in an amount between about 1-30% w/w of the formulation; and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/v sodium hydroxide solution, or a combination thereof. In some embodiments, the detergent portion comprises glycerin in an amount less than 3% w/w of the formulation.

In the presence of a polar gelling agent, such as water, glycerol, ethylene glycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about 1% w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Additional components that can also be included in a transdermal delivery formulation are fatty acids, terpenes, lipids, and cationic, and anionic detergents. In some embodiments, a transdermal delivery formulation further comprises tranexamic acid in an amount less than 2% w/w, 5% w/w, or 10% w/w of the formulation. In some embodiments, a transdermal delivery formulation further comprises a polar solvent in an amount less than 2% w/w, 5% w/w, 10% w/w, or 20% w/w of the transdermal delivery formulation. In some embodiments, a transdermal delivery formulation further comprises a humectant, an emulsifier, an emollient, or a combination thereof. In some embodiments, a transdermal delivery formulation further comprises almond oil in an amount less than about 5% w/w. In some embodiments, a formulation further comprises a mixture of thermoplastic polyurethane and polycarbonate in an amount less than about 5% w/w. In some embodiments, a transdermal delivery formulation further comprises phosphatidylethanolamine in an amount less than about 5% w/w. In some embodiments, a transdermal delivery formulation further comprises an inositol phosphatide in an amount less than about 5% w/w.

Other solvents and related compounds that can be used in some embodiments include acetamide and derivatives, acetone, n-alkanes (chain length between 7 and 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, d-limonene, N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C₃-C₆ triols or their mixtures and a polar lipid compound selected from C₁₆ or C₁₈ monounsaturated alcohol, C₁₆ or C₁₈ branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that can be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monooleate, sorbitan trilaurate, sorbitan trioleate, sucrose coconut fatty ester mixtures, sucrose monolaurate, sucrose monooleate, tetradecyl N.N-dimethylamino acetate. Examples of suitable fatty acid·include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include a-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxypropyl glycerol).

Examples of completing agents that can be used in some embodiments include β- and γ-cyclodextrin complexes, hydroxypropyl methylcellulose (e.g., Carbopol® 934), liposomes, naphthalene diamide diimide, and naphthalene diester diimide.

One or more antioxidants can be included, such as vitamin C, vitamin E, proanthocyanidin and a-lipoic acid typically in concentrations of 0.1%-2.5% w/w.

In some applications, it is desirable to adjust the pH of a transdermal delivery formulation to assist in permeation or to adjust the nature of the target compounds in the subject. In some instances, the pH is adjusted to a level of pH 9-11, pH 7, pH 8, pH9, pH10, pH 11, pH12 or pH10-11 which can be done by providing appropriate buffers or simply adjusting the pH with base.

A transdermal delivery formulation can include other components that act as excipients or serve purposes other than for treating psoriasis. For example, preservatives like antioxidants e.g., ascorbic acid or α-lipoic acid and antibacterial agents can be included. Other components apart from therapeutically active ingredients and components that are the primary effectors of dermal penetration can include those provided for aesthetic purposes such as menthol or other aromatics, and components that affect the physical state of the composition such as emulsifiers, for example, polyglyceryl-4 monolaurate. Typically, these ingredients are present in small percentages of the compositions. It is understood that these latter ancillary agents are neither therapeutic ingredients nor are they components that are primarily responsible for penetration of the skin. The components that primarily effect skin penetration have been detailed as described above. However, some of these substances have some capability for effecting skin penetration. See, for example, Kunta, J. R. et al, J. Pharm. Sci. (1997) 86:1369-1373, describing penetration properties of menthol.

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of a transdermal delivery formulation itself. The application of a transdermal delivery formulation may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a transdermal delivery formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In addition to the compositions and formulations of the invention per se, the methods can employ a subsequent treatment with linoleic acid. As transdermal treatments generally open up the skin barrier, which is, indeed, their purpose, it is useful to seal the area of application after the treatment is finished. Thus, treatment with a transdermal delivery formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow the active component to pass through the epidermis to the dermis at least, and, if systemic administration is achieved, through the dermis itself.

Additional therapeutic agents can be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol can be included in an amount ranging from about 0.1% w/w to about 1.0% w/w.

In some particular embodiments it is desirable to adjust the pH of a transdermal delivery formulation and the pH is adjusted to a level of pH 9-11 or 10-11, which can be done by providing appropriate buffers or simply adjusting the pH with base. In other embodiments, it is desirable to adjust the pH of a transdermal delivery formulation to a level of pH 4-6, which can be done by providing appropriate buffers or simply adjusting the pH with an acid.

In some applications a formulation for transdermal delivery may, for example, comprise: Aveeno®, for example in an amount between about 10-95% w/w; between about 20-85% w/w, between about 20-75% w/w, between about 20-50% w/w.

In another aspect, certain embodiments are directed to a sustained release drug delivery platform releases a therapeutic compound or compounds disclosed and made as a formulation described herein over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release drug delivery platform releases a therapeutic compound or compounds disclosed herein with substantially first order release kinetics over a period of, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

The formulation described in this specification may also comprise more than one therapeutic compound as desired for the particular indication being treated, preferably those with complementary activities that do not adversely affect the other proteins. A transdermal delivery formulation to be used for in vivo administration can be sterile. This can be accomplished, for instance, without limitation, by filtration through sterile filtration membranes, prior to, or following, preparation of a transdermal delivery formulation or other methods known in the art, including without limitation, pasteurization.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

In certain embodiments, kits can comprise, without limitation, one or more cream or lotion comprising one or more formulations described herein. In various embodiments, the kit can comprise of formulation components for transdermal, topical, or subcutaneous administration, formulated to be administered as an emulsion coated patch. In all of these embodiments and others, the kits can contain one or more lotion, cream, patch, or the like in accordance with any of the foregoing, wherein each patch contains a single unit dose for administration to a subject.

Imaging components can optionally be included, and the packaging also can include written or web-accessible instructions for using a transdermal delivery formulation. A container can include, for example, a vial, bottle, patch, syringe, pre-filled syringe, tube or any of a variety of formats well known in the art for multi-dispenser packaging.

### Methods

Methods for treating, preventing or ameliorating a disease, disorder, a condition, or a symptom thereof or a condition related thereto are provided herein using a transdermal delivery formulation for transdermal delivery described herein below. The methods provided herein may comprise or consist of topically administering one or more of a transdermal delivery formulation described herein to skin of a subject in need thereof. Preferred, but non-limiting embodiments are directed to methods for treating, preventing, inhibiting or ameliorating a disease, disorder, a condition, or a symptom described below.

An approach to make electrolyte balancing formulations is to avoid electrolyte imbalances by incorporating different buffers in different amount or ratios. Non-limiting examples of buffering agents that can be used together in different amounts or ratios include potassium bicarbonate, sodium bicarbonate, calcium carbonate, magnesium carbonate, and potassium carbonate. Mixtures of particular buffering agents including 2, 3, 4, 5, or more buffering agents are used depending on the formulation. Further, the relative amounts or ratio of each buffering agent may vary, for example, where the relative amounts are from 1:1.10 w/w; 1:1.15 w/w; 1:1.20 w/w; 1:1.25 w/w; 1:1.30 w/w; 1:1.35 w/w; 1:1.40 w/w; 1:1.45 w/w; 1:1.50 w/w; 1:1.55 w/w; 1:1.60 w/w; 1:1.65 w/w; 1:1.70 w/w; 1:1.75 w/w; 1:1.80 w/w; 1:1.85 w/w; 1:1.90 w/w; 1:1.95 w/w; 1:2 w/w; 1:2.5 w/w; 1:3 w/w; 1:3.5 w/w; 1:4 w/w, 1:4.5 w/w; 1:5 w/w, 1:5.5 w/w; 1:6 w/w; 1:6.5 w/w; 1:7 w/w; 1:8 w/w; 1:9 w/w; or 1:10 w/w. These ratios of buffering agents are applicable when two buffering agents are present, or more than two and the ratios are applicable between any two buffering agents.

### Formulations

A formulation for transdermal delivery can, for example, comprise two components or it may comprise one or more buffering agent and a penetrant. Typically, however, a penetrant is less than 85% w/w. A transdermal delivery formulation may have a detergent of at least 1% w/w. For example, a suitable formulation may comprise about 10-56% w/w buffering agent and a penetrant. In one aspect, disclosed herein is a transdermal delivery formulation for transdermal delivery of one or more buffering agent through the skin of a subject, comprising: a buffering agent comprising a carbonate salt in an amount between about 10-56% w/w; a transdermal delivery formulation in an amount between about 5 to 55% w/w; a detergent portion in an amount of at least 1% w/w; and wherein the formulation comprises water in an amount from none up to about 77% w/w.

In an embodiment, a carbonate, including sodium bicarbonate in a transdermal delivery formulation is in an amount of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more w/w.

In another embodiment, a buffering agent comprising a carbonate salt, including sodium bicarbonate in a transdermal delivery formulation is in an amount of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more w/w.

In yet another embodiment, disclosed herein is a formulation for transdermal delivery of a therapeutic agent through the skin of a subject, wherein the formulation comprises at least one active agent in an amount effective for treatment of a condition in the subject and the formulation comprising: a buffering agent comprising a carbonate salt in an amount between about 10-45% w/w; a transdermal delivery formulation in an amount between about 5 to 55% w/w; a detergent portion in an amount between about 1 to 15% w/w; wherein the formulation comprises water in an amount between about 15 to 65% w/w, through the skin of a subject, wherein the carbonate salt of the formulation is in an amount between about 15-32% w/w of the formulation, therapeutic, and wherein the alkalinity of the formulation enhances penetration of the therapeutic agent.

In In yet another aspect, disclosed herein is a formulation for transdermal delivery of cyclosporine through the skin of a subject, wherein the formulation comprises at least cyclosporine in an amount effective for treatment of a condition in the subject and the formulation comprising: a buffering agent in an amount between about 1-45% w/w; a transdermal delivery formulation in an amount between about 5 to 55% w/w; a detergent portion in an amount between about 1 to 15% w/w; wherein the formulation comprises water in an amount between about 15 to 65% w/w, through the skin of a subject, and wherein the formulation comprises less than about 12% w/w of the transdermal delivery formulation.

In some embodiments, a transdermal delivery formulation comprises: Aveeno® in an amount between about 20-85% w/w, or in an amount of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more w/w.

In some embodiments, a suitable transdermal delivery formulation comprises: water in an amount between about 10-55% w/w; isopropyl palmitate in an amount between about 0.5-10% w/w; stearic acid in an amount between about 0.25-5% w/w; cetyl alcohol in an amount between about 0.25-10% w/w; almond oil in an amount between about 0.5-10% w/w; propylene glycol in an amount between about 0.25-10% w/w; ethanol in an amount less than about 5% w/w; and benzyl alcohol in an amount less than about 5% w/w.

The surprising effects achieved by the formulations and methods of the present invention are in part attributable to an improved transdermal delivery formulation that enhances delivery of a cyclosporinet through the skin. The present transdermal delivery formulations may include a nonionic surfactant. Applicant has found that by employing cyclosporine as disclosed herein, delivered with the penetrants as disclosed herein, and in some embodiments providing a combination of a nonionic surfactant and a polar gelling agent, the penetration capabilities of the cyclosporine of the resulting formulation and the effective level of delivery of the has been enhanced.

In a transdermal delivery formulation, penetrants are based on combinations of an alcohol, such as benzyl alcohol to provide a concentration of 0.5-20% w/w of the final formulation with a transdermal delivery formulation present to provide 25-70% w/w of the formulation. These penetrants are also useful when the agent is cyclosporine, but less of a transdermal delivery formulation may be required—e.g. less than 12% w/w when the sodium bicarbonate is present at high concentration as disclosed herein.

Alternatively, the penetrant component comprises a completion component as well as one or more electrolytes sufficient to impart viscosity and viscoelasticity, one or more surfactants and an alcohol. The completion component can be a polar liquid, a non-polar liquid or an amphiphilic substance.

A transdermal delivery formulation of the disclosure may be prepared in a number of ways. Typically, the components of a transdermal delivery formulation are simply mixed together in the required amounts. However, it is also desirable in some instances to, for example, carry out dissolution of cyclosporine and then add a separate preparation containing the components aiding the delivery of the cyclosporine in the form of a carrier. The concentrations of these components in the carrier, then, will be somewhat higher than the concentrations required in a final transdermal delivery formulation. Thus, cyclosporine may first be dissolved in water and then added to a carrier comprising an alcohol, a transdermal delivery formulation and optionally a combination of a nonionic surfactant and polar gelling agent, or of ionic detergent. Alternatively, some subset of these components can first be mixed and then “topped off” with the remaining components either simultaneously or sequentially. The precise manner of preparing a transdermal delivery formulation will depend on the cyclosporine and the percentages of the remaining components that are desirable with respect to that cyclosporine. In some embodiments, the water is in an amount between about 10-85% w/w, 15-50% w/w, or 15-45% w/w of the formulation.

The transdermal delivery formulation is a multi-component mixture, whereby the particular concentrations of the penetration enhancers are informed in part by the molecular mass of the cyclosporine to be transported. A transdermal delivery formulation enables the cyclosporine to become bio-available to the target site within minutes of topical administration. A transdermal delivery formulation permit the use of minimal concentrations of cyclosporine, as little as 1/1000th of concentrations required of alternative processes, while enabling bioactivity and positive clinical outcomes simultaneously. In some embodiments, the transdermal delivery formulation comprises an alcohol in an amount less than 5% w/w of the formulation.

### Administration and Dosing

A transdermal delivery formulation provided herein can be topically administered in any form. For administration for the treatment of skin conditions a sufficient amount of the topical composition can be applied onto a desired area and surrounding skin, for example, in an amount sufficient to cover a desired skin surface. A transdermal delivery formulation can be applied to any skin surface, including for example, facial skin, and the skin of the hands, neck, chest and/or scalp.

In applying a transdermal delivery formulation of the invention, a transdermal delivery formulation itself is simply placed on the skin and spread across the surface and/or massaged to aid in penetration. The amount of transdermal delivery formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of a transdermal delivery formulation to the skin and prevents distortion of a transdermal delivery formulation by evaporation in some cases. The composition may be applied to the skin using standard procedures for application such as a brush, a syringe, a gauze pad, a dropper, or any convenient applicator. More complex application methods, including the use of delivery devices, may also be used, but are not required.

In an alternative to administering topically to intact skin, the surface of the skin may also be disrupted mechanically by the use of spring systems, laser powered systems, use of iontophoresis, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed transdermal delivery formulations that penetrate intact skin may be applied using occlusive patches, such as those in the form of micro-patches. External reservoirs of the formulations for extended administration may also be employed.

Accordingly, in certain embodiments alternative methods of administering one or more buffering agent, therapeutic compounds, agents, drugs through intact skin are provided. As nonlimiting examples, these alternative methods might be selected from the following lists: on basis of working mechanism, spring systems, laser powered, energy-propelled, Lorentz force, gas/air propelled, shock wave (including ultrasound), on basis of type of load, liquid, powder, projectile, on basis of drug delivery mechanism, nano-patches, sandpaper (microdermabrasion), iontophoresis enabled, microneedles, on basis of site of delivery, intradermal, intramuscular, and subcutaneous injection. Other suitable delivery mechanisms include, without limitation, microneedle drug delivery, such as 3M Systems, Glide SDI (pushes drug as opposed to “firing” drug), MIT low pressure injectors, micropatches (single use particle insertion device), microelectro mechanical systems (MEMS), dermoelectroporation devices (DEP), transderm ionto system (DEP), TTS transdermal therapeutic systems, membrane-moderated systems (drug reservoir totally encapsulated in a shallow compartment), adhesive diffusion-controlled system (drug reservoir in a compartment fabricated from drug-impermeable metallic plastic backing), matrix dispersion type system (drug reservoir formed by homogeneously dispersing drug solids in a hydrophilic or lipophilic polymer matrix molder into medicated disc), and microreservoir system (combination of reservoir and matrix dispersion-type drug delivery system).

The application method is determined by the nature of the treatment but may be less critical than the nature of a transdermal delivery formulation itself. If the application is to a skin area, it may be helpful in some instances to prepare the skin by cleansing or exfoliation. In some instances, it is helpful to adjust the pH of the skin area prior to application of the formulation itself. The application of a transdermal delivery formulation may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a transdermal delivery formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In addition to a transdermal delivery formulation of the invention per se, the methods may employ a subsequent treatment with linoleic acid. As transdermal treatments generally open up the skin barrier, which is, indeed, their purpose, it is useful to seal the area of application after the treatment is finished. Thus, treatment with a transdermal delivery formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow active ingredients to pass through the epidermis to the dermis at least, and, if systemic administration is achieved, through the dermis itself.

Additional therapeutic agents may be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol may be included in an amount ranging from about 0.1% w/w to about 1.0% w/w.

A transdermal delivery formulation can be applied in a single, one-time application, once a week, once a bi-week, once a month, or from one to twelve times daily, for a period of time sufficient to alleviate a condition, disease, disorder, symptoms, for example, for a period of time of one week, from 1 to 12 weeks or more, from 1 to 6 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be topically administered once or more per day for a period of time from 1 week to 4 weeks, of from 1 week to 2 weeks, for 1 week, for 2 weeks, for 3 weeks, or for 4 weeks or more. In some instances, it may also be desirable to continue treatment indefinitely for example to inhibit or prevent signs and symptoms of psoriasis. A suitable administration for a transdermal delivery formulation comprising a skin cream, lotion or ointment, for example is once, twice, three, four times daily, or hourly if needed.

As described above, if desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A transdermal delivery formulation in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the transdermal delivery formulation may be prepared. Single dose, unit dose, and once-daily disposable containers of the transdermal delivery formulation are also provided.

The present transdermal delivery formulation remains stable in storage for periods including up to about 5 years, between about 3 months and about 5 years, between about 3 months and about 4 years, between about 3 months and about 3 years, and alternately any time period between about 6 months and about 3 years.

A transdermal delivery formulation described herein remains stable for up to at least 3 years at a temperature of less than or equal to 10° C., less than or equal to 15° C., less than or equal to 20° C., less than or equal to 25° C., less than or equal to 30° C., less than or equal to 35° C., less than or equal to 40° C. In an embodiment, the presently described transdermal delivery formulation remains stable for at least 2 years at a temperature of less than or equal to 10° C., less than or equal to 15° C., less than or equal to 20° C., less than or equal to 25° C., less than or equal to 30° C., less than or equal to 35° C., less than or equal to 40° C. In an embodiment, the presently described transdermal delivery formulation remains stable for at least 3 years at a temperature of less than or equal to 10° C., less than or equal to 15° C., less than or equal to 20° C., less than or equal to 25° C., less than or equal to 30° C., less than or equal to 35° C., less than or equal to 40° C. and at a humidity of up to 5% RH, up to 10% RH, up to 15% RH, up to 20% RH, up to 25% RH, up to 30% RH, up to 35% RH, up to 40% RH, up to 45% RH, up to 50% RH, up to 55% RH, up to 60% RH, up to 65% RH, up to 70 or up to 75% RH, for at least 2 years at a temperature of less than or equal to 10° C., less than or equal to 15° C., less than or equal to 20° C., less than or equal to 25° C., less than or equal to 30° C., less than or equal to 35° C., less than or equal to 40° C. and at a humidity of up to 5% RH, up to 10% RH, up to 15% RH, up to 20% RH, up to 25% RH, up to 30% RH, up to 35% RH, up to 40% RH, up to 45% RH, up to 50% RH, up to 55% RH, up to 60% RH, up to 65% RH, up to 70 or up to 75% RH, or for at least 3 years at a temperature of less than or equal to 30° C. and at a humidity of up to 75% RH. In a further embodiment, the presently described transdermal delivery formulation in accordance with the subject matter described herein remains stable for an extended period of time when packaged in a multi-use container such as a bottle dispenser or the like, and exhibits equal to or even greater stability when packaged in a single-use package.

In another aspect, the transdermal delivery formulation of certain embodiments comprises a daily dose of cyclosporine. A daily dose for topical or transdermal administration of a transdermal delivery formulation depends on the compound and animal and may be easily determined by the skilled artisan, a suitable amount is about 1 mg/kg to about 5 g/kg, and more typically the daily dose is about 10 mg/kg to about 5 g/kg, about 25 mg/kg to about 2000 mg/kg, about 50 mg/kg to about 2000 mg/kg, about 25 mg/kg to about 1000 mg/kg, about 50 mg/kg to about 1000 mg/kg, about 100 mg/kg to about 700 mg/kg, about 100 mg/kg to about 500 mg/kg, about 150 mg/kg to about 500 mg/kg, about 150 mg/kg to about 400 mg/kg, about 200 mg/kg to about 500 mg/kg, about 200 mg/kg to about 450 mg/kg, about 200 mg/kg to about 400 mg/kg, about 250 mg/kg to about 450 mg/kg, about 250 mg/kg to about 400 mg/kg, about 250 mg/kg to about 350 mg/kg, and about 275 mg/kg to about 325 mg/kg.

Alternatively, a suitable daily dose for a transdermal delivery formulation of cyclosporine is at least about 1 mg/kg, at least about 10 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 90 mg/kg, at least about 100 mg/kg, at least about 125 mg/kg, at least about 150 mg/kg, at least about 160 mg/kg, at least about 170 mg/kg, at least about 175 mg/kg, at least about 180 mg/kg, at least about 190 mg/kg, at least about 200 mg/kg, at least about 225 mg/kg, at least about 250 mg/kg, at least about 275 mg/kg, at least about 300 mg/kg, at least about 325 mg/kg, at least about 350 mg/kg, at least about 375 mg/kg, at least about 400 mg/kg, at least about 425 mg/kg, at least about 450 mg/kg, at least about 475 mg/kg, at least about 500 mg/kg, at least about 550 mg/kg, at least about 600 mg/kg, at least about 700 mg/kg, at least about 800 mg/kg, at least about 900 mg/kg, at least about 1 g/kg, at least about 2 g/kg, at least about 3 g/kg, or at least about 5 g/kg. In an embodiment, the cyclosporine concentration is at least 0.5 mg/kg, at least 0.75 mg/kg, at least 1 mg/kg, at least 1.5 mg/kg, at least 2 mg/kg, at least 2.5 mg/kg, at least 3 mg/kg, at least 3.5 mg/kg, at least 4 mg/kg, at least 4.5 mg/kg, at least 5 mg/kg, at least 5.5 mg/kg, at least 6 mg/kg, at least 6.5 mg/kg, at least 7 mg/kg, at least 7.5 mg/kg, at least 8 mg/kg, at least 8.5 mg/kg, at least 9 mg/kg, at least 9.5 mg/kg, at least 10 mg/kg. In an embodiment, the cyclosporine concentration is no more than 0.5 mg/kg, no more than 0.75 mg/kg, no more than 1 mg/kg, no more than 1.5 mg/kg, no more than 2 mg/kg, no more than 2.5 mg/kg, no more than 3 mg/kg, no more than 3.5 mg/kg, no more than 4 mg/kg, no more than 4.5 mg/kg, no more than 5 mg/kg, no more than 5.5 mg/kg, no more than 6 mg/kg, no more than 6.5 mg/kg, no more than 7 mg/kg, no more than 7.5 mg/kg, no more than 8 mg/kg, no more than 8.5 mg/kg, no more than 9 mg/kg, no more than 9.5 mg/kg, no more than 10 mg/kg. In an embodiment, the cyclosporine concentration is about 0.5 mg/kg, about 0.75 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A transdermal delivery formulation in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the transdermal delivery formulation may be prepared. Single dose, unit dose, and once-daily disposable containers of the transdermal delivery formulation are also provided.

Alternatively, a suitable dose for topical or transdermal administration of cyclosporine) for subject is at least about 0.1 mg, at least about 0.25 mg, at least about 0.5 mg, at least about 0.75 mg, at least about 1 mg, at least about 1.5 mg, at least about 2 mg, at least about 2.5 mg, at least about 3 mg, at least about 3.5 mg, at least about 4 mg, at least about 4.5 mg, at least about 5 mg, at least about 5.5 mg, at least about 6 mg, at least about 6.5 mg, at least about 7 mg, at least about 7.5 mg, at least about 8 mg, at least about 89.5 mg, at least about 9 mg, at least about 9.5 mg, at least about 10 mg, at least about 20 mg, at least about 25 mg, at least about 30 mg, at least about 40 mg, at least about 50 mg, at least about 60 mg, at least about 70 mg, at least about 80 mg, at least about 90 mg, at least about at least about 100 mg, at least about 500 mg, at least about 1 g, at least about 5 g, at least about 10 g, at least about 15 g, at least about 16 g, at least about 17 g, at least about 18 g, at least about 19 g, at least about 20 g, at least about 21 g, at least about 22 g, at least about 23 g, at least about 24 g, at least about 25 g, at least about 26 g, at least about 27 g, at least about 28 g, at least about 29 g, at least about 30 g, at least about 35 g, at least about 40 g, at least about 45 g, at least about 50 g, at least about 60 g, at least about 75 g, at least about 100 g, at least about 200 g, at least about 500 g, or at least about 1.0 kg. This dose may be administered daily, twice a day, three times a day, four times a day, five times a day, or more than five times a day.

Aspects of the present specification disclose that the symptoms associated with a disease or disorder described herein are reduced following application of a transdermal delivery formulation by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced following application of a transdermal delivery formulation by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another aspect, in certain embodiments a pH modulating transdermal delivery formulation (e.g. containing sodium bicarbonate) is administered topically or transdermally with the cyclosporine, wherein the dose results in a subject intake of at least about 0.1 nmol/hr/Kg, at least about 0.5 nmol/hr/Kg, at least about 0.7 nmol/hr/Kg, at least about 1.0 nmol/hr/Kg, at least about 1.1 nmol/hr/Kg, at least about 1.2 nmol/hr/Kg, at least about 1.3 nmol/hr/Kg, at least about 1.4 nmol/hr/Kg, at least about 1.5 nmol/hr/Kg, at least about 1.6 nmol/hr/Kg, at least about 1.7 nmol/hr/Kg, at least about 1.8 nmol/hr/Kg, at least about 1.9 nmol/hr/Kg, at least about 2.0 nmol/hr/Kg, at least about 2.5 nmol/hr/Kg, at least about 3.0 nmol/hr/Kg, at least about 3.5nmol/hr/Kg, at least about 4.0 nmol/hr/Kg, at least about 5 nmol/hr/Kg, at least about 10 nmol/hr/Kg, at least about 25 nmol/hr/Kg, at least about 50 nmol/hr/Kg, at least about 100 nmol/hr/Kg, at least about 500 nmol/hr/Kg, or at least about 1 μmol/hr/Kg of cyclosporine.

A transdermal delivery formulation as described herein can be used in the manufacture of medicaments and for the treatment of humans and other animals by administration in accordance with conventional procedures.

Dosing can be single dosage or cumulative (serial dosing), and can be readily determined by one skilled in the art. A transdermal delivery formulation of the present invention may be administered once, twice, three, four, five, six, seven, eight, nine, ten, eleven, twelve, thirteen, fourteen, fifteen, sixteen, seventeen, eighteen, nineteen, twenty or more times to a subject. For instance, treatment of a disease may comprise a one-time administration of an effective dose of a transdermal delivery formulation as disclosed herein. Alternatively, treatment of a disease may comprise multiple administrations of an effective dose of a transdermal delivery formulation as carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a transdermal delivery formulation as disclosed herein can be administered to an individual once daily for an indefinite period of time, or until the individual no longer requires therapy. A person of ordinary skill in the art will recognize that the condition of the individual can be monitored throughout the course of treatment and that the effective amount of a transdermal delivery formulation disclosed herein that is administered can be adjusted accordingly. In one embodiment, a transdermal delivery formulation as disclosed herein is capable of decreasing the time to resolve the symptoms of a disease, including in an individual suffering from a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In a further embodiment, an anti-psoriasis transdermal delivery formulation and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of an anti-psoriasis transdermal delivery formulation is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of an anti-psoriasis transdermal delivery formulation disclosed herein reduces or alleviates symptoms of psoriasis in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of an anti-psoriasis transdermal delivery formulation disclosed herein reduces or alleviates symptoms by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of an anti-psoriasis transdermal delivery formulation disclosed herein reduces or alleviates symptoms by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A transdermal delivery formulation disclosed herein may comprise an anti-psoriasis transdermal delivery formulation in a therapeutically effective amount. As used herein, the term “effective amount” is synonymous with “therapeutically effective amount”, “effective dose”, or “therapeutically effective dose” and when used in reference to reducing or alleviate symptoms of psoriasis to achieve the desired therapeutic effect and includes a dose sufficient to reduce or alleviate signs and symptoms of psoriasis. The effectiveness of an anti-psoriasis delivery formulation disclosed herein capable of reducing or alleviating symptoms in an individual can be determined by observing an improvement in an individual based upon one or more clinical symptoms, and/or physiological indicators associated with reducing symptoms such as inflammation and skin irritation in an individual. The effectiveness of anti-psoriasis transdermal delivery formulation disclosed herein is also capable of enhancing the quality of life of an individual as compared to the same individual if the anti-psoriasis transdermal delivery formulation is not administered.

The appropriate effective amount of an anti-psoriasis transdermal delivery formulation disclosed herein to be administered can be determined by a person of ordinary skill in the art by taking into account factors, including, without limitation, the area of inflammation or patches observed on the individual, amount of itching/discomfort, or any combination thereof. Additionally, where repeated administration of a transdermal delivery formulation is used, an effective amount will further depend upon factors, including, without limitation, the frequency of administration, the half-life of the anti-psoriasis transdermal delivery formulation, or any combination thereof. It is known by a person of ordinary skill in the art that an effective amount of an anti-psoriasis transdermal delivery formulation disclosed herein can be extrapolated from in vitro assays and in vivo administration studies using animal models prior to administration to humans or animals.

Wide variations in the necessary effective amount are to be expected in view of the differing efficiencies of the various routes of administration. For instance, oral administration of a transdermal delivery formulation disclosed herein generally would be expected to require higher dosage levels than administration by inhalation. Similarly, systemic administration of a transdermal delivery formulation disclosed herein would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a therapeutic disclosed herein that is administered can be adjusted accordingly.

Aspects of the present specification disclose, in part, reduction or alleviation of psoriasis in an individual. As used herein, the term “treating,” refers to reduction or alleviation of symptoms in an individual. For example, the term “treating” can mean reduction or alleviation of symptoms in an individual by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with psoriasis, including itchiness, inflammation and red patches on the skin and are well known and can be determined by a person of ordinary skill in the art by using commonly known testing means. Those of skill in the art will know the appropriate symptoms or indicators associated with psoriasis and will know how to determine if an individual is a candidate for treatment as disclosed herein.

In an embodiment, a first anti-psoriasis transdermal delivery formulation is administered to an individual and at a later date, a second anti-psoriasis transdermal delivery formulation is administered to the same individual. In an embodiment, a first anti-psoriasis transdermal delivery formulation is administered to an individual at the same time as a second anti-psoriasis transdermal delivery formulation is administered to the individual.

In one aspect, disclosed herein is a formulation for transdermal delivery of cyclosporine with or without a therapeutic agent through the skin, nail or hair follicle of a subject, wherein the formulation comprises one or more of the following a) a transdermal delivery formulation in an amount less than about 60% w/w, comprising i. one or more phosphatides, ii. carbohydrate, and iii. one or more fatty acids; and b) water in an amount less than about 50% w/w.

In some embodiments, the formulation, which herein is a cyclosporine formulation with or without a therapeutic agent comprises one or more of the following a) a transdermal delivery formulation in an amount less than about 60% w/w, comprising i. one or more phosphatides, ii. carbohydrate, and iii. one or more fatty acids; and b) water in an amount less than about 50% w/w, further comprises benzyl alcohol in an amount between about 0.5-5% w/w.

In some embodiments, the transdermal delivery formulation, which herein is a cyclosporine formulation with or without a therapeutic agent comprises benzyl alcohol in an amount less than 5% w/w of the formulation.

In some embodiments, the formulation, which herein is a cyclosporine formulation with or without a therapeutic agent comprises one or more of the following a) a transdermal delivery formulation in an amount less than about 60% w/w, comprising i. one or more phosphatides, ii. carbohydrate, and iii. one or more fatty acids; and b) water in an amount less than about 50% w/w, further comprises isopropyl palmitate in an amount between about 5-20% w/w.

In some embodiments, the water is deionized water and/or purified water.

In some embodiments, the water is in an amount between about 15-40% w/w of the formulation.

In some embodiments, the one or more phosphatides in an amount between about 0.5-55% w/w of the transdermal delivery formulation.

In some embodiments, the transdermal delivery formulation, which herein is a cyclosporine formulation with or without a therapeutic agent comprises phosphatidylcholine, hydrogenated phosphatidylcholine, phosphatidylserine, phosphatidylethanolamine, phosphatidylinositol, or a combination thereof in amount less than 30% w/w of the formulation.

In some embodiments, the one or more phosphatides comprises phosphatidylcholine of the transdermal delivery formulation.

In some embodiments, the one or more fatty acids in an amount between about 1-35% w/w of the transdermal delivery formulation.

In some embodiments, the one or more fatty acids in an amount between about 5-35% w/w of the transdermal delivery formulation.

In some embodiments, the one or more fatty acids comprises linoleic acid, oleic acid, stearic acid, sunflower oil, or a combination thereof.

In some embodiments, the one or more fatty acids comprises linoleic acid.

In some embodiments, the one or more fatty acids comprises oleic acid.

In some embodiments, the one or more fatty acids comprises stearic acid.

In some embodiments, the one or more phosphatides are derived from a seed oil in an amount between about 0.5-55% w/w of the transdermal delivery formulation.

In some embodiments, the one or more phosphatides are derived from a seed oil in an amount between about 5-35% w/w of the transdermal delivery formulation.

In some embodiments, the one or more phosphatides are derived from a safflower oil in an amount between about 0.5-55% w/w of the transdermal delivery formulation.

In some embodiments, the one or more phosphatides are derived from a safflower oil in an amount between about 5-35% w/w of the transdermal delivery formulation.

In some embodiments, the one or more phosphatides are derived from an almond oil in an amount between about 0.5-55% w/w of the transdermal delivery formulation.

In some embodiments, the one or more phosphatides are derived from an almond oil in an amount between about 0.5-10% w/w of the transdermal delivery formulation. In another embodiment, the amount of almond oil is about 1%, about 2%, about 3%, about 4%, about 5%, about 6%, about 7%, about 8%, about 9%, about 10%, or more. In a further embodiment, the amount of almond oil is no more than 1%, no more than 2%, no more than 3%, no more than 4%, no more than 5%, no more than 6%, no more than 7%, no more than 8%, no more than 9%, no more than 10%. In a further embodiment, the amount of almond oil is less than 1%, less than 2%, less than 3%, less than 4%, less than 5%, less than 6%, less than 7%, less than 8%, less than 9%, less than 10%.

In some embodiments, the one or more phosphatides comprises one or more fatty acids derived from soy lecithin.

In some embodiments, the carbohydrate in an amount between about 0.05-10% w/w of the transdermal delivery formulation.

In some embodiments, the carbohydrate is anhydrous dextrose in an amount between about 0.05-10% w/w of the transdermal delivery formulation.

In some embodiments, the formulation, which herein is a cyclosporine formulation with or without a therapeutic agent comprises one or more of the following a) a transdermal delivery formulation in an amount less than about 60% w/w, comprising i. one or more phosphatides, ii. carbohydrate, and iii. one or more fatty acids; and b) water in an amount less than about 50% w/w, further comprises a nonionic surfactant in an amount between about 2-25% w/w of the transdermal delivery formulation.

In some embodiments, the formulation, which herein is a cyclosporine formulation with or without a therapeutic agent comprises one or more of the following a) a transdermal delivery formulation in an amount less than about 60% w/w, comprising i. one or more phosphatides, ii. carbohydrate, and iii. one or more fatty acids; and b) water in an amount less than about 50% w/w, further comprises a polar solvent at least in an amount in molar excess of the nonionic surfactant.

In some embodiments, the nonionic surfactant is a poloxamer and the polar solvent is water.

In some embodiments, the formulation, which herein is a cyclosporine formulation with or without a therapeutic agent comprises one or more of the following a) a transdermal delivery formulation in an amount less than about 60% w/w, comprising i. one or more phosphatides, ii. carbohydrate, and iii. one or more fatty acids; and b) water in an amount less than about 50% w/w, further comprises a polar solvent in an amount less than 5% w/w of the formulation.

In some embodiments, the transdermal delivery formulation, which herein is a cyclosporine formulation with or without a therapeutic agent further comprises a detergent portion in an amount between about 1-30% w/w of the transdermal delivery formulation.

In some embodiments, the detergent portion comprises a nonionic surfactant in an amount between about 2-25% w/w of the transdermal delivery formulation; and a polar solvent in an amount less than 5% w/w of the transdermal delivery formulation.

In some embodiments, the transdermal delivery formulation, which herein is a cyclosporine formulation with or without a therapeutic agent is in an amount between about 10-60% w/w of the transdermal delivery formulation.

In some embodiments, the transdermal delivery formulation comprises an alcohol in an amount less than 10% w/w of the transdermal delivery formulation.

In some embodiments, the transdermal delivery formulation further comprises an alcohol, a surfactant, and a polar solvent.

In some embodiments, the transdermal delivery formulation, which herein is a cyclosporine formulation with or without a therapeutic agent comprises cetyl alcohol in amount less than 5% w/w of the formulation.

In some embodiments, the transdermal delivery formulation, which herein is a cyclosporine formulation with or without a therapeutic agent comprises ethanol in an amount less than 5% w/w of the formulation.

In some embodiments, the transdermal delivery formulation, which herein is a cyclosporine formulation with or without a therapeutic agent comprises glycerin in an amount less than 5% w/w of the formulation.

In some embodiments, the transdermal delivery formulation, which herein is a cyclosporine formulation with or without a therapeutic agent comprises propylene glycol in an amount less than 8% w/w of the formulation.

In some embodiments, the formulation, which herein is a cyclosporine formulation with or without a therapeutic agent comprises a gelling agent in an amount less than 20% w/w of the formulation.

In some embodiments, the formulation, which herein is a cyclosporine formulation with or without a therapeutic agent comprises menthol in an amount between about 0.05-5% w/w of the formulation.

In some embodiments, the formulation, which herein is a cyclosporine formulation with or without a therapeutic agent comprises one or more of the following a) a transdermal delivery formulation in an amount less than about 60% w/w, comprising i. one or more phosphatides, ii. carbohydrate, and iii. one or more fatty acids; and b) water in an amount less than about 50% w/w.

In some embodiments, the formulation, which herein is a cyclosporine formulation with or without a therapeutic agent comprises a) a transdermal delivery formulation in an amount less than about 60% w/w, comprising i. one or more phosphatides, ii. carbohydrate, and iii. one or more fatty acids; and b) water in an amount less than about 50% w/w, further comprises a humectant, an emulsifier, an emollient, or a combination thereof.

In some embodiments, the formulation, which herein is a cyclosporine formulation with or without a therapeutic agent, which herein is a cyclosporine formulation with or without a therapeutic agent has a pH of about 2, about 3 about 4, about 4.2, about 4.5, about 5 about 6 about 7, about 8, about 9, about 10, about 11, about 12. In some embodiments, the formulation, which herein is a cyclosporine formulation with or without a therapeutic agent, which herein is a cyclosporine formulation with or without a therapeutic agent has a pH of less than 2, less than 3 less than 4, less than 4.2, less than 4.5, less than 5 less than 6 less than 7, less than 8, less than 9, less than 10, less than 11, less than 12. In some embodiments, the formulation, which herein is a cyclosporine formulation with or without a therapeutic agent, which herein is a cyclosporine formulation with or without a therapeutic agent has a pH of at least 2, at least 3 at least 4, at least 4.2, at least 4.5, at least 5 at least 6 at least 7, at least 8, at least 9, at least 10, at least 11, at least 12. In another embodiment, the formulation, which herein is a cyclosporine formulation with or without a therapeutic agent, which herein is a cyclosporine formulation with or without a therapeutic agent has a pH of in a range of 1-5, in a range of 2-5, in a range of 3-5, in a range of 4-5, in a range of 3 to 11, in a range of 4 to 11, in a range of 3 to 10, in a range of 4 to 10, in a range of 3 to 9, in a range of 4 to 9, in a range of 3 to 8, in a range of 4 to 8, in a range of 3 to 7, in a range of 4 to 7, in a range of 3 to 6, in a range of 4 to 6, in a range of 3 to 5 or in a range of 4 to 5.

In some embodiments, the formulation, which herein is a cyclosporine formulation with or without a therapeutic agent, which herein is a cyclosporine formulation with or without a therapeutic agent has a pH of about 2, about 3 about 4, about 4.2, about 4.5, about 5 about 6 about 7, about 8, about 9, about 10, about 11, about 12. In some embodiments, the formulation, which herein is a cyclosporine formulation with or without a therapeutic agent, which herein is a cyclosporine formulation with or without a therapeutic agent has a pH of less than 2, less than 3 less than 4, less than 4.2, less than 4.5, less than 5 less than 6 less than 7, less than 8, less than 9, less than 10, less than 11, less than 12. In some embodiments, the formulation, which herein is a cyclosporine formulation with or without a therapeutic agent, which herein is a cyclosporine formulation with or without a therapeutic agent has a pH of at least 2, at least 3 at least 4, at least 4.2, at least 4.5, at least 5 at least 6 at least 7, at least 8, at least 9, at least 10, at least 11, at least 12. In another embodiment, the formulation, which herein is a cyclosporine formulation with or without a therapeutic agent, which herein is a cyclosporine formulation with or without a therapeutic agent has a pH of in a range of 1 to 5, in a range of 2 to 5, in a range of 3 to 5, in a range of 4 to 5, in a range of 3 to 11, in a range of 4 to 11, in a range of 3 to 10, in a range of 4 to 10, in a range of 3 to 9, in a range of 4 to 9, in a range of 3 to 8, in a range of 4 to 8, in a range of 3 to 7, in a range of 4 to 7, in a range of 3 to 6, in a range of 4 to 6, in a range of 3 to 5 or in a range of 4 to 5.

In some embodiments, the formulation, which herein is a cyclosporine formulation with or without a therapeutic agent comprises one or more of the following a) a transdermal delivery formulation in an amount less than about 60% w/w, comprising i. one or more phosphatides, ii. carbohydrate, and iii. one or more fatty acids; and b) water in an amount less than about 50% w/w, further comprises an active agent.

In some embodiments, the formulation, which herein is a cyclosporine formulation with or without a therapeutic agent comprises one or more of the following a) a transdermal delivery formulation in an amount less than about 60% w/w, comprising i. one or more phosphatides, ii. carbohydrate, and iii. one or more fatty acids; and b) water in an amount less than about 50% w/w, further comprises an active agent component in an amount less than about 60% w/w.

In another aspect disclosed herein is a method to effect transdermal delivery of an active ingredient comprising applying to the skin, nails or hair follicles of a subject an effective amount of the formulation comprising one or more of the following a) a transdermal delivery formulation in an amount less than about 60% w/w, comprising i. one or more phosphatides, ii. carbohydrate, and iii. one or more fatty acids; and b) water in an amount less than about 50% w/w, further comprises an active agent.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Murine Pharmacokinetics Study

In this experiment, the absorption of cyclosporine was studied in mice treated with a transdermal formulation. 2% Cyclosporine was incorporated into the topical formulation referenced in Table 1. This formulation was applied to mice at 100 mg/mouse in a pharmacokinetic (PK) study.

At multiple time points, blood samples were taken from the mice and analyzed for cyclosporine concentration (ng/ml). The results are presented in Table 2. The data was used to calculate the values in Table 3. Notable values include Half-life (T½), peak plasma concentration (Cmax), and time to reach peak plasma concentration (Tmax).

TABLE 2

| Time | | Mean | SD | SE | |
|---|---|---|---|---|---|
| (h) | N | (ng/ml) | (ng/ml) | (ng/ml) | CV % |
| 0.5 | 3 | 736 | 791 | 457 | 107.5 |
| 1 | 3 | 613 | 288 | 166 | 46.9 |
| 2 | 3 | 704 | 138 | 79.7 | 19.6 |
| 4 | 3 | 425 | 261 | 150 | 61.4 |
| 8 | 3 | 595 | 151 | 87.2 | 25.4 |
| 24 | 3 | 57.2 | 21.1 | 12.2 | 36.9 |

TABLE 3

| K_el | T1/2 | Tmax | Cmax | AUClast | AUCinf | MRTlast |
|---|---|---|---|---|---|---|
| (1/h) | (h) | (h) | (ng/ml) | (h*ng/ml) | (h*ng/ml) | (h) |
| 0.11 | 6.21 | 0.5 | 736 | 9566 | 10079 | 7.0 |

As can be seen in Table 3, the study showed a Cmax of 736 ng/ml, Tmax of 0.5 h, and T½ of 6.21 h. This high concentration in a short time of onset is unique in topical cyclosporine. In a separate study that treated cats with topical cyclosporine daily for 21 days, the highest mean concentration was 58 ng/ml which was measured 2 hrs after application on the 21st day.

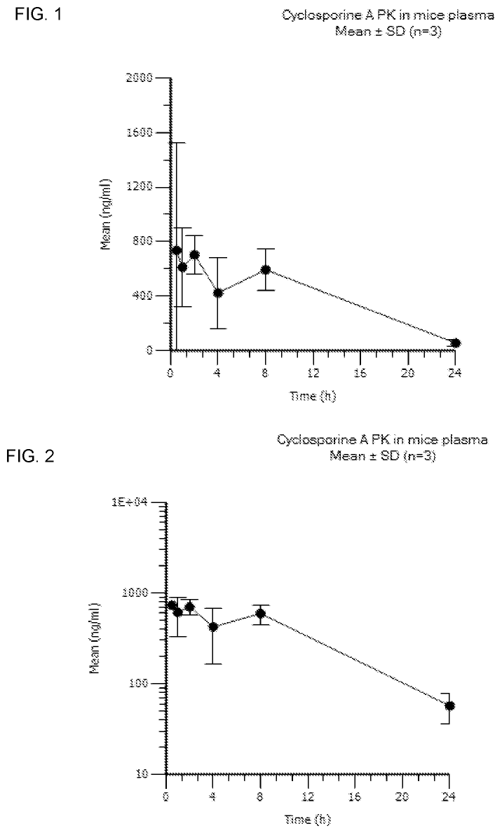

FIGS. 1 and 2. PK data showing resulting mean plasma cyclosporine concentration, measured at 0.5, 1, 2, 4, 8, and 24 hours.

### Example 2

### Use of Topical Cyclosporine for Treatment of Psoriasis

In this example, a patient is affected with psoriasis. The signs and symptoms of psoriasis are not alleviated with conventional treatments, including topical steroidal compounds.

The patient can use a topical cream, as described herein, that contains cyclosporine. The lotion can be applied regularly (e.g. daily) to areas with itching or other symptoms. Alternatively, the cream can be applied based on needs or circumstances. For example, the cream can be applied when the patient anticipates or notices inflammation or itchiness. The cream can be applied more generously with more intense or outbreaks.

The lotion or cream can include a transdermal delivery formulation with cyclosporine. In this example, the dose of the active agent (i.e. cyclosporine) is 3 grams so that it is 2-5% of the solution. The transdermal delivery formulation can include less than about 60% w/w of one or more phosphatides, one or more fatty acids and water. The lotion/cream can be used to treat local inflammation from psoriasis and used, for example, over a 24-hour period or until symptoms are adequately reduced.

### Example 3

### Coadministration of Topical Cyclosporine for Treatment of Psoriasis

In this example, a patient affected with psoriasis that is unresponsive to conventional steroidal compounds. The patient can use a topical cream, as described herein, that contains cyclosporine and one or more additional active agents. Such active agents can include a vitamin D and/or an agent to reduce inflammation. Other active agents can include vitamin D analogues (i.e. synthetic vitamin D), anthralin, topical retinoids (derived from vitamin A), topical calcineurin inhibitors and immunosuppressants.

The lotion/cream can be used to periodically to prevent outbreaks of psoriasis. For example. the lotion can be applied regularly (e.g. daily) to areas that are prone to itching or redness from psoriasis. The dose of the active agent (i.e. cyclosporine) is 3 grams so that it is 2-5% of the solution. The transdermal delivery formulation can include less than about 60% w/w of one or more phosphatides, one or more fatty acids and water.

### Example 4

### Coadministration of Topical Cyclosporine for Treatment of Eczema

In this example, a patient affected with eczema that is unresponsive to conventional steroidal compounds. The patient can use a topical cream, as described herein, that contains cyclosporine and one or more additional active agents. Such active agents can include a vitamin D and/or an agent to reduce inflammation. Other active agents can include vitamin D analogues (i.e. synthetic Vitamin D), anthralin, topical retinoids (derived from Vitamin A), and topical calcineurin inhibitors.

The lotion/cream can be used to periodically to relieve eczema and/or prevent outbreaks. For example. the lotion can be applied regularly (e.g. daily) to areas that are prone to itching or redness from eczema. The dose of the active agent (i.e. cyclosporine) is 3 grams so that it is 2-5% of the solution. The transdermal delivery formulation can include less than about 60% w/w of one or more phosphatides, glucose, one or more fatty acids and water.

### Example 5

### Administration of Topical Cyclosporine for Treatment of Rheumatoid Arthritis

Rheumatoid arthritis (RA) is a long-term autoimmune disorder that primarily affects joints. Conventional treatments include pain medication, steroids and NSAID's. In this example, a patient experiences joint swelling and soreness from RA that is unresponsive to conventional treatments. The patient can use a topical cream, as described herein, that contains cyclosporine and optionally one or more additional active agents such as a NSAID.

The lotion/cream can be used to periodically to relieve and/or prevent symptoms such as swelling and soreness. For example. the lotion can be applied regularly (e.g. daily) to affected areas such as knuckles and joints. The dose of the active agent (i.e. cyclosporine) is 3 grams so that it is 2-5% of the solution. The transdermal delivery formulation can include less than about 60% w/w of one or more phosphatides, one or more fatty acids and water.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A transdermal delivery formulation comprising the following components:
a. cyclosporine at a concentration from 0.5% to 5.0%;
b. isopropyl palmitate at a concentration from 5% to 20%;
c. benzyl alcohol at a concentration of 0.5% to 5%;
d. stearic acid at a concentration from 0.5% to 5%;
e. safflower oil at 1% to 6%;
f. oleic acid at 0.5% to 2%; and
g. deionized water at 20% to 80%;

2. The transdermal delivery formulation of claim 1, wherein the transdermal formulation also includes:
a. Aveeno® moisturizers, cream, oils, lotions;
b. Jergens® moisturizers, cream, oils, lotions;
c. Honest Company® moisturizers, cream, oils, lotions;
d. Dermologica® moisturizers, cream, oils, lotions; or
e. St. Ives™ moisturizers, cream, oils, lotions

3. The transdermal delivery formulation of claim 1, wherein the transdermal formulation further comprises phosphatidylcholine at a concentration of 1% to 20%.

4. The transdermal delivery formulation of claim 1, wherein the transdermal formulation further comprises polyglyceryl-4 laurate at a concentration of 0.5% to 5%.

5. The transdermal delivery formulation of claim 1, wherein the transdermal formulation further comprises 30% Pluronic Gel (mixture of water and poloxamer 407) at a concentration of 5% to 40%.

6. The transdermal delivery formulation of claim 1, wherein the transdermal formulation also includes a surfactant.

7. The transdermal delivery formulation of claim 1, wherein the transdermal formulation also includes a nonionic detergent.

8. The transdermal delivery formulation of claim 1, wherein the transdermal formulation also includes a polar gelling agent.

9. The transdermal delivery formulation of claim 7, wherein the nonionic detergent results in a more viscous and cream-like formulation.

10. The transdermal delivery formulation of claim 8, wherein the polar gelling agent results in a more viscous and gel-like formulation

11. The transdermal delivery formulation of claim 1, wherein the cyclosporin concentration is from 0.05% to 0.1%, from 0.1% to 0.5%, from 0.5% to 2%, from 0.5% to 1.5%, from 1% to 1.5%, from 0.5% to 1.5%, from 1% to 2.5%, from 1% to 3%, from 1.5% to 3%, from 1% to 4% or from 1% to 5%.

12. The transdermal delivery formulation of claim 1, wherein the isopropyl palmitate is from 1% to 15%, from 2.5% to 15%, from 4% to 15%, from 5% to 10%, from 10% to 15%, from 12% to 15%, from 5% to 8%, from 5% to 15% or from 10% to 20%.

13. The transdermal delivery formulation of claim 1, wherein the benzyl alcohol is from 0.5% to 1.5%, 0.5% to 4%, from 0.75% to 3%, from 1% to 2.5%, from 2% to 4% or from 2.5% to 5%.

14. The transdermal delivery formulation of claim 1, wherein the stearic acid is from 0.5% to 1.5%, from 1.5% to 2.5%, from 3.5% to 5%, from 2% to 5%, from 3% to 5% or from 4% to 5%.

15. The transdermal delivery formulation of claim 1, wherein the safflower oil is at a concentration from 1% to 3%, from 1.5% to 2.5%, from 3% to 5% from 4 to 6%, from 4.5% to 6% or from 5% to 6%.

16. The transdermal delivery formulation of claim 15, wherein the safflower oil is a linoleic acid.

17. The transdermal delivery formulation of claim 1, wherein the oleic acid is at a concentration from 0.5% to 1%, from 0.5% to 1.5%, from 1% to 1.5% or from 1% to 2%.

18. The transdermal delivery formulation of claim 1, wherein the deionized water is from 20% to 50%, from 25% to 75%, from 30% to 60%, from 40% to 60%, from 40% to 50%, or from 50% to 80%.

19. A method of administration of cyclosporine to an individual using the transdermal delivery formulation of claim 1.

20. A method of treatment of psoriasis using the transdermal delivery formulation of claim 1.

21. The method of claim 20, wherein the psoriasis is at least one of plaque psoriasis, guttate psoriasis, inverse psoriasis, pustular psoriasis or erythrodermic psoriasis.

22. The transdermal delivery formulation of claim 1, wherein the formulation includes one or more additional agents selected from vitamin D, vitamin D analogues (i.e. synthetic vitamin D), anthralin, topical retinoids (derived from vitamin A), and topical calcineurin inhibitors.

23. A method of treatment of a skin condition using the transdermal delivery formulation of claim 1, wherein the skin condition is at least one of dermatitis, poison ivy and poison oak, and drug rashes, acne, cold sore, hives, keratosis, rosacea, carbuncle, eczema or cellulitis.

24. A method of treatment of an autoimmune condition using the transdermal delivery formulation of claim 1, wherein the autoimmune condition is at least one of dermatitis, lupus erythematosus, rheumatoid arthritis, celiac disease, diabetes mellitus type 1, Graves' disease, inflammatory bowel disease, multiple sclerosis, rheumatoid arthritis, chronic or non-specific inflammation, Guillain-Barre syndrome, chronic inflammatory demyelinating polyneuropathy, Hashimoto's thyroiditis, myasthenia gravis, vasculitis, fibromyalgia, Crohn's disease, myasthenia gravis, scleritis, vasculitis or systemic lupus erythematosus.
